(19) United States
(12) Patent Application Publication
(10) Pub. No.: US 2003/0133903 A1
(43) Pub. Date: Jul. 17, 2003

(54) Compositions for treatment of prostate cancers and methods of making and using the same

(76) Inventors: Wenbin Dang, Belle Mead, NJ (US); Rena Lapidus, Pikesville, MD (US); William Vincek, Baltimore, MD (US)

Correspondence Address: FOLEY HOAG LLP, PATENT GROUP, WORLD TRADE CENTER WEST, 155 SEAPORT BOULEVARD, BOSTON, MA, 02110-2600, US

(21) Appl. No.: 10/200,040

(22) Filed: Jul. 19, 2002

Related U.S. Application Data
(60) Provisional application No. 60/306,537, filed on Jul. 19, 2001.

(51) Int. Cl. A61K 31/74; A61K 31/337

(52) U.S. Cl. 424/78.17; 514/449

(57) ABSTRACT

The present invention relates to compositions of a biocompatible polymer and an antineoplastic agent, and methods of using and making the same, for the treatment of prostate cancers. In certain embodiments, the polymer contains phosphorous linkages.

### RELATED APPLICATION INFORMATION

This application claims the benefit of priority to Provisional Patent Application No. 60/306,537, filed Jul. 19, 2001, which application is hereby incorporated by reference in its entirety.

### INTRODUCTION

I. Background and Description of Related Art

Prostate cancer is the most common cancer, excluding non-melanoma skin cancers, in American men. The American Cancer Society estimates that in the year 2000 approximately 180,400 new cases of prostate cancer will be diagnosed in the United States. Prostate cancer is the second leading cause of cancer death in men, exceeded only by lung cancer. Prostate cancer causes about 11 percent of all cancer deaths in men. Furthermore, it is estimated that approximately 5 million men have at this very moment a histological cancer of the prostate, which may or may not ever become clinically evident. The prostate gland is about the size of a walnut and is located in front of the rectum, under the bladder and surrounds the upper part of the urethra. It contains gland cells that produce a portion of the seminal fluid which protects and nourishes sperm cells. Although other cells exist in the prostate, over 99% of prostate cancers develop from glandular cells; the tumors are termed adenocarcinomas.

Treatment options for prostate cancer depend upon its extent in the patient. The extensiveness of a malignancy is traditionally described by a system of stages, with higher stages indicating more extensive disease and thus decreased survival. A common staging system for many cancers is the TMN system. According to this system, the extent of a malignant disease is graded according to its tumor size (T), the number of involved lymph nodes (N), and the presence or absence of distant metastases (M). For prostate cancer, the TNM system has been combined with the well-established categories proposed initially by Whitmore in 1956 and subsequently modified. (Whitmore W F, “Natural history strategy of prostate cancer,” Urol. Clin. North Am. 11:205, 1984). In Whitmore's scheme, Stage I included clinically latent prostate cancer, Stage II included clinically manifest early prostate cancer, Stage III included clinically manifest locally advanced prostate cancer, and Stage IV included advanced prostate cancer with evidence of distant metastasis. The current staging system for prostate cancer looks at tumor size from T1 (clinically unapparent tumor not palpable or visible by imaging), to T2 (tumor confined within the prostate) to T3 (tumor extending through the prostate capsule) to T4 (tumor fixed or invading adjacent structures), along with the presence or absence of regional lymph node involvement (N0 or N1) and the presence or absence of metastatic disease (M0 or M1). (1997 American Joint Committee on Cancer/International Union against Cancer TNM staging classification, in Fleming I D, Cooper J S, et al., Manual for staging of cancer, Philadelphia: Lippincott-Raven, 1997). Studies are carried out prior to initiating therapy to determine the clinical stage of the disease. Based on these studies a stage is determined for a given patient's disease, upon which treatment options are based.

Patients diagnosed with a clinically localized or “early-stage” prostate cancer may be treated with surgery, radiation, local ablation, or by non-treatment or “watchful waiting.” Surgery is usually used for locally confined disease and is usually curative. There are three types: radical prostatectomy, transurethal resection of the prostate (TURP) and cryosurgery. These procedures are invasive, possess significant side effects (urinary incontinence and impotence), require hospital stays and time out of work. Definitive surgical treatment to extirpate the early stage cancer often involves some type of radical prostatectomy. (Catalona W J et al., “Contemporary results of anatomic radical prostatectomy,” CA Cancer J Clin 40: 282, 1999). Modifications of surgical techniques have been developed to preserve potency in patients undergoing radical prostatectomy. (Ruckle H C & Zincke H, “Potency-sparing radical retropubic prostatectomy: a simplified anatomical approach,” J. Urol. 153: 1875, 1995). Meticulous surgical technique is vital, however, to minimizing the incidence of positive surgical margins and consequent recurrent disease. (Rosen M A et al., “Frequency and location of extracapsular extension and positive surgical margins in radical prostatectomy specimens,” J. Urol. 148: 331, 1992).

Alternatively, radiation treatment may be elected. Radiation therapy uses high energy rays (gamma rays or x-rays) and particles (electrons, protons or neutrons) to kill cancer cells. Radiation is used to treat cancers that are confined to the prostate or have spread to nearby tissues. If the disease is more advanced, radiation may be used to reduce the size of the primary tumor to alleviate symptoms. Patients who have radiation therapy usually do not require surgery unless the radiation was ineffective. Two main types of radiation are external beam radiation and brachytherapy. With either of these treatment modalities in the clinically localized prostate cancer (T1 or T2 lesions), good long-term survival is expected, and progression of the disease as measured by serum prostate-specific antigen (PSA) levels appears to be well controlled in about 80% of patients.

External beam radiation is focused from a source outside the body on the area affected by the cancer. Patients are usually treated 5 days per week for 7 to 8 weeks. The procedure itself is painless but the extended side effects include bowel problems (diarrhea, rectal leakage and colitis), bladder problems (frequent urination) and impotence. There are two somewhat new forms of external beam radiation that appear promising in increasing the success rate and reducing the side effects. Three-dimensional conformal radiation therapy and conformal proton beam radiation therapy involve using sophisticated computers to map the location of the cancer. The radiation beams are then aimed from several directions. Because these procedures are relatively new, their impact on long term survival is unknown. Short term results suggest that by aiming the radiation more accurately, less normal tissue is damaged and there is improved efficacy by increasing the radiation dose to the cancer.

Brachytherapy or internal radiation therapy uses small radioactive pellets (each about the size of a grain of rice) that are implanted into the prostate. The radioactive materials (¹²⁵I, ¹⁰³Pd, etc.) are placed inside thin needles which are inserted through the skin of the perineum into the prostate (imaging systems such as transrectal ultrasound, CT or MRI are used to guide placement of seeds). The pellets give off radiation for weeks or months and the pellets are left in place after the radioactive material is used up. Side effects of brachytherapy may include impotence, urinary incontinence and bowel problems but these occur less frequently than with external beam radiotherapy and surgery.

Certain factors can be clinically identified that correlate with worsening prognosis. Larger tumors, extension of disease outside the prostate capsule or into the seminal vesicles, and poorly differentiated histopathology are all signs that tumor extirpation alone, whether by radiation or surgery, is likely to be durably successful. (Epstein J I, et al., “Correlation of pathologic findings with progression after radical retropubic prostatectomy,” Cancer 71:3582, 1993). Treatment failure, characterized by local recurrence or distant metastasis, is more likely in patients with such adverse indicators. Prostate cancer extending beyond the capsule of the prostate on clinical staging is considered locally advanced. (Epstein J I, “Evaluation of radical prostatectomy capsular margins of resection. The significance of margins designated as negative, closely approaching, and positive,” Am. J. Surg. Pathol. 14:626, 1990). Such tumors are not uncommon, comprising about 15 to 20 percent of newly diagnosed prostate cancers. In these patients, treatment goals include control of local disease, decrease of local morbidity and risk of recurrence, and prevention of distant metastases with associated prolongation of survival. Traditionally, control of local disease for the locally advanced prostate cancer patient has been provided by radiation treatment rather than surgery. In locally advanced prostate cancer, though, there may be a significant incidence of local recurrence in those patients initially treated successfully with radiation. (Schild S E, “Radiation therapy (RT) after prostatectomy: The case for salvage therapy as opposed to adjuvant therapy,” Int. J. Cancer 96: 94, 2001). As an alternative, surgical excision of the locally advanced cancer by radical prostatectomy with or without pelvic lymphadenectomy is also available, although it, too, is accompanied by a significant risk of local relapse. In certain cases, surgical treatment may be supplemented by immediate adjuvant irradiation. (Vallicenti R K & Gomella L G, “Durable efficacy of adjuvant radiation therapy for prostate cancer: will the benefit last?,” Semin. Urol. Oncol. 18: 115, 2000). Any of these local treatments may be combined with systemic treatment, most commonly androgen ablation.

The presence of regional lymph node metastasis presents an even more worrisome prognostic sign. (Cheng L, et al., “Risk of prostate carcinoma death in patients with lymph node metastasis,” Cancer 91: 66, 2001). Because they have already traveled to the regional lymph nodes, the cells of the adenocarcinoma originating in the prostate have already demonstrated their ability and propensity to spread beyond the prostate itself. Hence, a question exists whether local measures, whether surgery or radiation, can in themselves provide a cure for the disease: involved regional lymph nodes may be a marker for systemic spread of the malignancy. It is understood that the presence of regional lymph node metastases at the time of initial therapy for presumably localized cancer indicates that the patient is at high risk for developing clinically significant distant metastases that may prove fatal. A controversial treatment issue relates to whether extended local treatment including regional lymphadenectomy will be successful alone in such a case, or whether a combined approach of local and systemic management should be initiated to prevent as-yet undiagnosed micrometastases from becoming established. Identifying those patients at greatest risk for having occult lymphatic involvement is important for determining in advance the type of treatment offered the patient. Fowler J E et al., “The incidence and extent of pelvic lymph node metastases in apparently localized prostatic cancer,” Cancer 47:2941, 1981). Pelvic lymph node dissection at the time of prostatectomy will provide a definitive answer about lymph node status, but raises the risk of postoperative complications. (Gingrich J R & Paulson D F, “The impact of PSA on prostate cancer management. Can we abandon routine staging pelvic lymphadenectomy?,” Surg. Oncol. Clin. N. Am. 4:335, 1995); Donohue R E et al., “Intraoperative and early complications of staging pelvic lymph node dissection in prostatic adenocarcinoma,” Urology 35:223, 1990). Studies have demonstrated associations between increased incidence of lymphatic involvement and more advanced clinical stage as well as higher pathological grade. (Osterling J E et al., “Correlation of clinical stage, serum prostatic acid phosphatase and preoperative Gleason grade with final pathological stage in 275 patients with clinically localized adenocarcinoma of the prostate,” J. Urol. 138: 92, 1987); Donohue R E, et al., “Prostatic carcinoma. Influence of tumor grade on results of pelvic lymphadenectomy,” Urology 17:435, 1981). Other prognostic markers, including acid phosphates level, DNA ploidy of the presenting lesion, and serum prostate-specific antigen (PSA) may also correlate with the likelihood of lymphatic disease. (Wu T T, et al., “Prediction of lymphatic spreading in prostatic cancer by prostate-specific antigen and Gleason's score,” Eur. Urol. 26:202, 1994). Since surgery that is undertaken in a patient with involve regional lymph nodes is not curative, such patients may be treated more satisfactorily with other approaches than extensive and aggressive local excision. Systemic therapy for these patients is generally initiated, including hormonal therapy or chemotherapy. Adjuvant radiation may be added to a standard radical prostatectomy in an effort to enhance local control of the disease.

Clinical local recurrence after adequate initial extirpation is a highly significant untoward event. (Ornstein D K, et al., “Evaluation and management of the man who has failed primary curative therapy for prostate cancer,” Urol. Clin. North Am. 25:591, 1998). Clinical recurrence is a harbinger of disease dissemination. (Kuban D A, et al., “Prognosis in patients with local recurrence after definite irradiation for prostatic carcinoma,” Cancer 63: 2421, 1989). In patients ultimately developing distant metastasis, those patients with local recurrence develop their metastatic disease sooner than those patients without local recurrence. It is hypothesized that microscopic recurrence precedes and is more frequent than clinical recurrence. Diagnosing microscopic recurrences generally requires a biopsy of the treated prostate bed. Early detection of local recurrence may permit more a more satisfactory salvage strategy to be carried out. If previous surgery has been performed, radiation is generally used to treat the local recurrence of disease. (Catton C et al., “Adjuvant and salvage radiation therapy after radical prostatectomy for adenocarcinoma of the prostate,” Radiother. Oncol. 59:51, 2001). If radiation was the initial treatment modality, salvage after local recurrence generally requires surgery, though other treatments such as cryotherapy may be employed in certain circumstances. (Gheiler E L et al., “Predictors for maximal outcome in patients undergoing salvage surgery for radio-recurrent prostate cancer,” Urology 47: 85, 1996). These salvage strategies, whether radiation, surgery or cryotherapy, are accompanied by a significant incidence of post-treatment complications, including impotence, incontinence, and local effects of radiation to pelvic tissues. (Vaida A & Soloway M S, “Salvage radical prostatectomy for radiorecurrent prostate cancer: morbidity revisited,” J. Urol. 164:1998, 2000).

Since normal prostate tissues depend on testicular androgens for growth, androgen deprivation has been used to impair the growth of prostate cancers. Androgen deprivation, through surgical or chemical means, has become a predominant mode of systemic therapy for prostate cancer. This treatment provides a mainstay for management of metastatic disease. There are several methods of treatment, for example, orchiectomy (removal of testicles), luteinizing hormone-release hormones, and anti-androgens.

As with other solid tumors, protocols have been established for prostate cancer to evaluate the usefulness of androgen ablation as a neoadjuvant therapy or as adjuvant therapy in certain cases, either to decrease risk of local recurrence or to diminish the possibility of distant metastasis following local extirpation of disease. (Mcleod D G & Kolvenbag G J, “Defining the role of antiandrogens in the treatment of prostate cancer,” Urology 47: (1A Suppl.) 85, 1996). Chemotherapy can also be used for patients whose prostate cancer has spread outside of the prostate gland. Chemotherapy may slow tumor growth or decrease pain.

Certain prostate cancers may be determined to be insensitive to androgen ablation therapy, perhaps because of somatic mutations in the gene encoding the androgen receptor in the cancer cells. In these cases, traditional chemotherapeutic agents have been used, either singly or in combination. Chemotherapy may provide some palliation for hormone-unresponsive metastatic disease, and may offer some relief when a patient relapses with hormone-unresponsive disease.

There remains a need in the art for methods to deliver drugs to treat prostate cancer. There also remains a need to enhance the efficacy of radiation treatment in this same group of tumors. In addition, there exists a need for methods to increase the effectiveness or surgical treatment of prostate cancer with proven locoregional disease or with high likelihood of locoregional disease. In those patients who undergo extensive and aggressive primary extirpative surgery, possibly including regional lymphadenectomy, there remains a need for reducing the likelihood of local recurrence and distant metastasis. Further, there remains a need for treatments to complement the availability of surgery or radiation in recurrent disease, recognizing the technical difficulties and risk of complications that accompany such salvage procedures. Finally, there exists an urgent need for developing treatment modalities to manage those prostate cancers refractory to androgen deprivation.

### SUMMARY OF THE INVENTION

It is an object of the invention to provide compositions and methods for introducing substances into the prostrate. In general such substances will be incorporated with a polymer that provides sustained release of the substance in vivo. In many embodiments, the substance will have therapeutic effects on a disease or condition affecting the prostrate. It is further understood that such substances may be administered as a sole treatment or in combination with surgical and/or other interventions, such as, for example, pharmacological treatments.

It is another object of the present invention to provide compositions and methods for the treatment of prostate cancer. In one aspect, the present invention may provide useful adjuvants for the treatment of those tumors at risk for local and distant failure following surgical extirpation, whether those tumors are treated by initial primary surgery or by initial primary radiation. It is a further object of the present invention to enhance the efficacy of treatment for prostate cancer where the disease involves the regional lymph nodes, or is likely to do so. In one aspect, the present invention may provide useful adjuvants for treating those tumors with demonstrated or prospective locoregional involvement so as to decrease the chances of local recurrence and/or distant metastasis. It is another object of the present invention to provide additional treatments to complement the therapies available for salvage of locoregional failure. In one aspect, the present invention may provide treatment modalities that enhance the effectiveness of available salvage methods or that permit salvage methods to be carried out with locoregional efficacy while minimizing complications. It is yet another object of the present invention to provide systems and methods for delivering a chemotherapeutic agent to a patient with locally advanced or metastatic prostate cancer so that systemic side effects are diminished. In one aspect, the present invention may be used in combination with other treatment modalities in certain embodiments. As examples, the systems and methods of the present invention may be used in conjunction with surgery, with radiation, with systemic chemotherapy or with a combination of these modalities.

In certain embodiments, electromagnetic radiations may be used to treat prostate cancer in conjunction with the subject compositions. The radiation treatment may be completed before, after, or concomitant with administration of a subject composition. As described in greater detail below, the order of radiation treatment may affect the results of any such therapies.

According to certain embodiments of the present invention, these objects and other desirable results may be accomplished by placing in the anatomic area being treated a therapeutically effective amount of a composition comprising a biocompatible, and optionally biodegradable, polymer and an antineoplastic agent suitable for such a disease. In certain practices of the present invention, the anatomic area being treated may be reached by an access device that conveys, transports, instills or delivers the composition of the present invention to the preselected anatomic location. In part, the present invention is directed to a polymer system for use in the above-described treatments, such as a biocompatible polymer, comprising an antineoplastic taxane, for example, paclitaxel, methods for treatment using the subject compositions, and methods of making and using the same.

In certain embodiments, a large percentage of the subject compositions may be an antineoplastic agent such as an antineoplastic taxane, that may be used to treat tumors of the prostate. For example, such an agent may comprise 5% to 60% or more of the subject composition, such as at least about 10%, at least about 30%, or at least about 50% of said agent.

In certain embodiments, administration of the subject polymers results in sustained release of an encapsulated antineoplastic agent for an extended period of time and in an amount that is not possible with other modes of administration. In certain embodiments, release of the antineoplastic agent follows zero order kinetics, i.e. the rate of release is independent of the concentration of antineoplastic agent present. In some instances there will be an initial burst, or higher rate of release, followed by a steady zero-order release. In one embodiment, the properties of the polymer: therapeutic complex are such that the burst is minimized.

The subject compositions, and methods of making and using the same, achieve a number of desirable results and features, one or more of which (if any) may be present in any particular embodiment of the present invention: (i) a single dose of a subject composition may achieve the desired therapeutically beneficial response to treat prostate cancers through sustained release of an antineoplastic agent; (ii) sustained release of an antineoplastic agent from a biocompatible and optionally biodegradable polymer composition in the prostate; (iii) novel treatment regimens for treating primary, recurrent or locally metastatic prostate cancer using the subject compositions for sustained delivery of an antineoplastic agent; (iv) high levels of loading (by weight), e.g. greater than 10% and up to 60% or more, of an antineoplastic agent for prostate cancer in biocompatible polymers; (v) lyophilization or subjection to an appropriate drying technique such as spray drying of the subject compositions and subsequent rehydration; and (vi) co-encapsulation of therapeutic agents in addition to any antineoplastic agent in biocompatible and optionally biodegradable polymers.

In one aspect, the subject polymers may be biocompatible, biodegradable or both. In certain embodiments, the subject polymers contain phosphorus linkages, including, for example, phosphate, phosphonate and phosphite. In other embodiments, the monomeric units of the present invention have the structures described in the claims appended below, which are hereby incorporated by reference in their entirety into this Summary. In the subject polymers, and in particular in those embodiments containing a phosphorus linkage, the chemical structure of certain of the monomeric units may be varied to achieve a variety of desirable physical or chemical characteristics, including for example, release profiles or handling characteristics of the resulting polymer composition.

In certain embodiments, other materials may be encapsulated in the subject polymer in the antineoplastic agent used to treat prostate cancer, to alter the physical and chemical properties of the resulting polymer, including for example, the release profile of the resulting polymer composition of such agent. Examples of such materials include biocompatible plasticizers, delivery agents, fillers and the like.

The compounds and methods of the present invention may be used in conjunction with antineoplastic agents administered locally or systemically according to dosage schedules known to the art or apparent to skilled artisans using no more than routine experimentation. Some possible antineoplastic agents that may be so used are described below.

The present invention provides a number of methods of making the subject compositions. Examples of such methods include those described in the Exemplification below.

In certain embodiments, the subject compositions are in the form of microspheres. In other embodiments, the subject compositions are in the form of nanospheres. In one aspect, the subject compositions of the present invention may be lyophilized or subjected to another appropriate drying technique such as spray drying and subsequently rehydrated for ready use.

In another aspect, the present invention is directed to methods of using the subject polymer compositions for prophylactic or therapeutic treatment of prostate cancer. In certain instances, the subject compositions may be used to prevent such a disease or condition. In certain embodiments, use of certain of the subject compositions, which release in a sustained manner an antineoplastic agent, allow for different treatment regimens for prostate cancer than are possible with other modes of administration of such an antineoplastic agent.

In another aspect of the invention, the efficacy of treatment using the subject compositions, optionally with electromagnetic radiation, may be compared to treatment regimens known in art in which an antineoplastic agent is not encapsulated within a subject polymer or other treatment regimens. For example, treatment with a subject composition is expected to result in fewer hypersensitivity reactions than treatment with an antineoplastic agent, such as paclitaxel, with or without premedication. Alternatively, treatment with a subject composition results in an increase in the median survival rate in mice, and it is expected that the same will result in other mammals, and in particular humans. Alternatively, the efficacy of treatment with a subject composition may be greater than with treatment with an antineoplastic agent alone or in a pharmaceutically acceptable carrier.

In another aspect, the subject polymers may be used in the manufacture of a medicament for any number of uses, including for example treating any disease or other treatable condition of a patient. In still other aspects, the present invention is directed to a method for formulating polymers of the present invention in a pharmaceutically acceptable carrier.

In another aspect, the present invention may be spray dried and subsequently rehydrated for ready use or injected as powder using appropriate powder injecting device.

In other embodiments, this invention contemplates a kit including subject compositions, and optionally instructions for their use. Uses for such kits include, for example, therapeutic applications. In certain embodiments, the subject compositions contained in any kit have been lyophilized and require rehydration before use.

The embodiments and practices of the present invention, other embodiments, and their features and characteristics, will be apparent from the description, drawings and claims that follow.

### BRIEF DESCRIPTION OF THE FIGURES

FIG. 1 depicts tumor reduction in mice after intratumoral administration of polymer microspheres containing paclitaxel.

FIG. 2 depicts tumor reduction in mice after subcutaneous administration of polymer microspheres containing paclitaxel.

FIG. 3 depicts reduction in PSA serum levels in mice after intratumoral administration of polymer microspheres containing paclitaxel.

FIG. 4 depicts reduction in PSA serum levels in mice after subcutaneous administration of polymer microspheres containing paclitaxel.

FIG. 5 depicts reduction in mice after intratumoral administration of administration of polymer microspheres containing paclitaxel, combined with radiation therapy.

FIG. 6 depicts reduction in mice after subcutaneous administration of administration of polymer microspheres containing paclitaxel, combined with radiation therapy.

### DETAILED DESCRIPTION OF THE INVENTION

1. Overview

The present invention relates to pharmaceutical compositions for the delivery of antineoplastic agents, including antineoplastic taxanes such as paclitaxel, for the treatment of prostate cancer. Treatment of prostate cancer includes treatment of local primary or recurrent disease, treatment of a site of extirpated disease, and treatment of the regional lymph nodes draining the primary disease site(i.e., the pelvic lymph nodes). A prostate cancer may comprise a primary tumor, i.e., one that affects a region of the prostate, a local recurrence, a regional metastasis or a distant matastasis (an “anatomic area”). A primary tumor that has spread to regional lymph nodes is understood to be regionally metastatic; a tumor that has spread to distant sites beyond the locoregional area is said to be distantly metastatic. In certain embodiments, biocompatible and optionally biodegradable polymers may be used to allow for sustained release of an encapsulated antineoplastic taxane to treat prostate cancer. The present invention also relates to methods of administering such pharmaceutical compositions, e.g., as part of a treatment regimen, for example, into tumors, into arteries or other vessels nourishing tumors, into an excised tumor bed, into the margins of an excised tumor, or in an anatomic area where a prostate cancer, either local or metastatic, has been identified. The present invention also provides for kits whereby said pharmaceutical compositions may be delivered to the aforesaid sites.

In certain aspects, the subject compositions, upon contact with body fluids including blood, lymph, tissue fluid or the like, release the encapsulated antineoplastic taxane over a sustained or extended period (as compared to the release from an isotonic saline solution). Such a system may result in prolonged delivery (over, for example, 2 to 4,000 hours, or even 4 to 1500 hours) of effective amounts (e.g., 0.00001 mg/kg/hour to 10 mg/kg/hour) of the drug. This dosage form may be administered as is necessary depending on the subject being treated, the severity of the affliction, the judgment of the prescribing physician, and the like.

For treatment of prostate cancers, the subject compositions of the present invention are adapted for application to a preselected anatomic area, for example an area of local disease or an area with regional metastasis or micrometastasis, or an area with a significant likelihood of containing residual disease after excision, or an area with a significant likelihood of bearing regional micrometastases. As used herein, the term “anatomic area” refers to any anatomic area that may be affected with a prostate cancer. In certain embodiments, the subject compositions of the present invention are understood to exert their effect in part by contact with a portion of the anatomic area being treated. Contact refers to a physical touching, either directly with the pharmaceutical composition being applied without intervening barrier to the anatomic area being treated, or indirectly, where the pharmaceutical composition is applied to or is formed on a surface of an interposed material, passing through to come into direct contact with the anatomic area being treated. Contact, as used herein, includes those situations where the pharmaceutical compounds of the present invention are initially positioned to contact the anatomic area being treated, and those situations where the pharmaceutical compounds of the present invention are initially positioned in proximity to the anatomic area being treated without contacting it, and subsequently move, migrate, flow, spread or are transported to enter into contact with the anatomic area being treated. Contact may include partial contacts, wherein the pharmaceutical compounds only contact a portion of the anatomic area being treated, or the edge or periphery or margin of the anatomic area being treated. Contact of the pharmaceutical compounds with the anatomic area being treated occurs from a local rather than systemic administration of said compounds, as these terms are defined hereinafter. The composition may be formed as a flowable material, insertable into the anatomic area. A variety of devices and methods for inserting the composition into the preselected anatomic area will be familiar to practitioners of ordinary skill in the art, for example infusion, injection, topical application, spraying, painting, coating, formed gel placement, and others. The composition, alternatively, may be formed as a solid object implantable in the anatomic area, or as a film or mesh that may be used to cover a segment of the area. A variety of techniques for implanting solid objects in relevant anatomic areas will be likewise familiar to practitioners of ordinary skill in the art.

In certain embodiments, the present invention may include the use of a composition in the manufacture of a medicament to treat prostate cancer, wherein said composition comprises a therapeutically effective amount of a composition comprising a biocompatible polymer and an antineoplastic agent appropriate for prostate cancer, wherein said biocompatible polymer comprises a biocompatible polymer having phosphorous-based linkages.

2. Definitions

For convenience, before further description of the present invention, certain terms employed in the specification, examples, and appended claims are collected here. These definitions should be read in light of the remainder of the disclosure and understood as by a person of skill in the art.

The articles “a” and “an” are used herein to refer to one or to more than one (i.e., to at least one) of the grammatical object of the article. By way of example, “an element” means one element or more than one element.

The term “access device” is an art-recognized term and includes any medical device adapted for gaining or maintaining access to an anatomic area. Such devices are familiar to artisans in the medical and surgical fields. An access device may be a needle, a catheter, a cannula, a trocar, a tubing, a shunt, a drain, or an endoscope such as a laparoscope, cystoscope, sigmoidoscope, or any other endoscope adapted for use in an anatomic area affected by a prostate cancer, or any other medical device suitable for entering or remaining positioned within the preselected anatomic area.

The terms “antineoplastic”, “antineoplastic agent” and “antineoplastic substance” are art-recognized, and refer to therapeutic agents that prevent the development, maturation, or spread of cells characterized by abnormal malignant growth, e.g., for treating or preventing prostrate cancer. Examples of antineoplastic agents are set forth below. In addition, one class of antineoplastic agents are antineoplastic taxanes, which are also defined in more detail below. In certain embodiments, an antineoplastic agent used in a composition of the invention to treat prostate cancer is as effective or more effective than paclitaxel or docetaxel, or is at least within an order of magnitude as effective as paclitaxel or docetaxel, e.g., has an ED₅₀ less than ten times the ED₅₀ of paclitaxel or docetaxel.

The terms “biocompatible polymer” and “biocompatibility” when used in relation to polymers are art-recognized. For example, biocompatible polymers include polymers that are neither themselves toxic to the host (e.g., an animal or human), nor degrade (if the polymer degrades) at a rate that produces monomeric or oligomeric subunits or other byproducts at toxic concentrations in the host. In certain embodiments of the present invention, biodegradation generally involves degradation of the polymer in an organism, e.g., into its monomeric subunits, which may be known to be effectively non-toxic. Intermediate oligomeric products resulting from such degradation may have different toxicological properties, however, or biodegradation may involve oxidation or other biochemical reactions that generate molecules other than monomeric subunits of the polymer. Consequently, in certain embodiments, toxicology of a biodegradable polymer intended for in vivo use, such as implantation or injection into a patient, may be determined after one or more toxicity analyses. It is not necessary that any subject composition have a purity of 100% to be deemed biocompatible; indeed, it is only necessary that the subject compositions be biocompatible as set forth above. Hence, a subject composition may comprise polymers comprising 99%, 98%, 97%, 96%, 95%, 90%, 85%, 80%, 75% or even less of biocompatible polymers, e.g., including polymers and other materials and excipients described herein, and still be biocompatible.

To determine whether a polymer or other material is biocompatible, it may be necessary to conduct a toxicity analysis. Such assays are well known in the art. One example of such an assay may be performed with live carcinoma cells, such as GT3TKB tumor cells, in the following manner: the sample is degraded in IM NaOH at 37° C. until complete degradation is observed. The solution is then neutralized with 1M HCl. About 200 1L of various concentrations of the degraded sample products are placed in 96-well tissue culture plates and seeded with human gastric carcinoma cells (GT3TKB) at 10⁴/well density. The degraded sample products are incubated with the GT3TKB cells for 48 hours. The results of the assay may be plotted as % relative growth vs. concentration of degraded sample in the tissue-culture well. In addition, polymers and formulations of the present invention may also be evaluated by well-known in vivo tests, such as subcutaneous implantations in rats to confirm that they do not cause significant levels of irritation or inflammation at the subcutaneous implantation sites.

The term “biodegradable” is art-recognized, and includes polymers, compositions and formulations, such as those described herein, that are intended to degrade during use. Biodegradable polymers typically differ from non-biodegradable polymers in that the former may be degraded during use. In certain embodiments, such use involves in vivo use, such as in vivo therapy, and in other certain embodiments, such use involves in vitro use. In general, degradation attributable to biodegradability involves the degradation of a biodegradable polymer into its component subunits, or digestion, e.g., by a biochemical process, of the polymer into smaller, non-polymeric subunits. In certain embodiments, two different types of biodegradation may generally be identified. For example, one type of biodegradation may involve cleavage of bonds (whether covalent or otherwise) in the polymer backbone. In such biodegradation, monomers and oligomers typically result, and even more typically, such biodegradation occurs by cleavage of a bond connecting one or more of subunits of a polymer. In contrast, another type of biodegradation may involve cleavage of a bond (whether covalent or otherwise) internal to side chain or that connects a side chain to the polymer backbone. For example, an antineoplastic taxane or other chemical moiety attached as a side chain to the polymer backbone may be released by biodegradation. In certain embodiments, one or the other or both generally types of biodegradation may occur during use of a polymer. As used herein, the term “biodegradation” encompasses both general types of biodegradation.

The degradation rate of a biodegradable polymer often depends in part on a variety of factors, including the chemical identity of the linkage responsible for any degradation, the molecular weight, crystallinity, biostability, and degree of cross-linking of such polymer, the physical characteristics of the implant, shape and size, and the mode and location of administration. For example, the greater the molecular weight, the higher the degree of crystallinity, and/or the greater the biostability, the biodegradation of any biodegradable polymer is usually slower. The term “biodegradable” is intended to cover materials and processes also termed “bioerodible”.

In certain embodiments, if the biodegradable polymer also has an antineoplastic taxane or other material associated with it, the biodegradation rate of such polymer may be characterized by a release rate of such materials. In such circumstances, the biodegradation rate may depend on not only the chemical identity and physical characteristics of the polymer, but also on the identity of any such material incorporated therein.

In certain embodiments, polymeric formulations of the present invention biodegrade within a period that is acceptable in the desired application. In certain embodiments, such as in vivo therapy, such degradation occurs in a period usually less than about five years, one year, six months, three months, one month, fifteen days, five days, three days, or even one day on exposure to a physiological solution with a pH between 6 and 8 having a temperature of between 25 and 37° C. In other embodiments, the polymer degrades in a period of between about one hour and several weeks, depending on the desired application.

The terms “comprise” and “comprising” are used in the inclusive, open sense, meaning that additional elements may be included.

The term “drug delivery device” is an art-recognized term and refers to any medical device suitable for the application of a drug or antineoplastic agent to a targeted organ or anatomic region. The term includes, without limitation, those formulations of the compositions of the present invention that release the antineoplastic agent into the surrounding tissues of an anatomic area. The term further includes those devices that transport or accomplish the instillation of the compositions of the present invention towards the targeted organ or anatomic area, even if the device itself is not formulated to include the composition. As an example, a needle or a catheter through which the composition is inserted into an anatomic area or into a blood vessel or other structure related to the anatomic area is understood to be a drug delivery device. As a further example, a stent or a shunt or a catheter that has the composition included in its substance or coated on its surface is understood to be a drug delivery device.

When used with respect to an antineoplastic agent or other material, the term “sustained release” is art-recognized. For example, a subject composition which releases a substance over time may exhibit sustained release characteristics, in contrast to a bolus type administration in which the entire amount of the substance is made biologically available at one time. For example, in particular embodiments, upon contact with body fluids including blood, tissue fluid, lymph or the like, the polymer matrices (formulated as provided herein and otherwise as known to one of skill in the art) may undergo gradual degradation (e.g., through hydrolysis) with concomitant release of any material incorporated therein, e.g., paclitaxel, for a sustained or extended period (as compared to the release from a bolus). This release may result in prolonged delivery of therapeutically effective amounts of any incorporated antineoplastic agent. Sustained release will vary in certain embodiments as described in greater detail below.

The term “delivery agent” is an art-recognized term, and includes molecules that facilitate the intracellular delivery of an antineoplastic agent or other material. Examples of delivery agents include: sterols (e.g., cholesterol) and lipids (e.g., a cationic lipid, virosome or liposome).

The terms “including” (and variants thereof), “such as”, “e.g.”, as used herein are non-limiting and are for illustrative purposes only. “Including” and “including but not limited to” are used interchangeably.

The term “microspheres” is art-recognized, and includes substantially spherical colloidal structures, e.g., formed from biocompatible polymers such as subject compositions, having a size ranging from about one or greater up to about 1000 microns. In general, “microcapsules”, also an art-recognized term, may be distinguished from microspheres, because microcapsules are generally covered by a substance of some type, such as a polymeric formulation. The term “microparticles” is art-recognized, and includes microspheres and microcapsules, as well as structures that may not be readily placed into either of the above two categories, all with dimensions on average of less than 1000 microns. If the structures are less than about one micron in diameter, then the corresponding art-recognized terms “nanosphere,” “nanocapsule,” and “nanoparticle” may be utilized. In certain embodiments, the nanospheres, nancapsules and nanoparticles have an average diameter of about 500, 200, 100, 50 or 10 nm.

A composition comprising microspheres may include particles of a range of particle sizes. In certain embodiments, the particle size distribution may be uniform, e.g., within less than about a 20% standard deviation of the median volume diameter, and in other embodiments, still more uniform or within about 10% of the median volume diameter.

The term “or” as used herein should be understood to mean “and/or”, unless the context clearly indicates otherwise.

The phrases “parenteral administration” and “administered parenterally” are art-recognized terms, and include modes of administration other than enteral and topical administration, such as injections, and include, without limitation, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

The term “treating” is art-recognized and includes preventing a disease, disorder or condition from occurring in an animal which may be predisposed to the disease, disorder and/or condition but has not yet been diagnosed as having it; inhibiting the disease, disorder or condition, e.g., impeding its progress; and relieving the disease, disorder or condition, e.g., causing regression of the disease, disorder and/or condition. Treating the disease or condition includes ameliorating at least one symptom of the particular disease or condition, even if the underlying pathophysiology is not affected.

The term “fluid” is art-recognized to refer to a non-solid state of matter in which the atoms or molecules are free to move in relation to each other, as in a gas or liquid. If unconstrained upon application, a fluid material may flow to assume the shape of the space available to it, covering for example, the surfaces of an excisional site or the dead space left under a flap. A fluid material may be inserted or injected into a limited portion of a space and then may flow to enter a larger portion of the space or its entirety. Such a material may be termed “flowable.” This term is art-recognized and includes, for example, liquid compositions that are capable of being sprayed into a site; injected with a manually operated syringe fitted with, for example, a 23-gauge needle; or delivered through a catheter. Also included in the term “flowable” are those highly viscous, “gel-like” materials at room temperature that may be delivered to the desired site by pouring, squeezing from a tube, or being injected with any one of the commercially available injection devices that provide injection pressures sufficient to propel highly viscous materials through a delivery system such as a needle or a catheter. When the polymer used is itself flowable, a composition comprising it need not include a biocompatible solvent to allow its dispersion within a body cavity. Rather, the flowable polymer may be delivered into the body cavity using a delivery system that relies upon the native flowability of the material for its application to the desired tissue surfaces. For example, if flowable, a composition comprising polymers according to the present invention it can be injected to form, after injection, a temporary biomechanical barrier to coat or encapsulate internal organs or tissues, or it can be used to produce coatings for solid implantable devices. In certain instances, flowable subject compositions have the ability to assume, over time, the shape of the space containing it at body temperature.

Viscosity is understood herein as it is recognized in the art to be the internal friction of a fluid or the resistance to flow exhibited by a fluid material when subjected to deformation. The degree of viscosity of the polymer may be adjusted by the molecular weight of the polymer and other methods for altering the physical characteristics of a specific polymer will be evident to practitioners of ordinary skill with no more than routine experimentation. The molecular weight of the polymer used in the composition of the invention may vary widely, depending on whether a rigid solid state (higher molecular weights) desirable, or whether a fluid state (lower molecular weights) is desired.

The phrase “pharmaceutically acceptable” is art-recognized. In certain embodiments, the term includes compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” is art-recognized, and includes, for example, pharmaceutically acceptable materials, compositions or vehicles, such as a liquid or solid filler, diluent, excipient, solvent or encapsulating material, involved in carrying or transporting any subject composition from one organ, or portion of the body, to another organ, or portion of the body. Each carrier must be “acceptable” in the sense of being compatible with the other ingredients of a subject composition and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “pharmaceutically acceptable salts” is art-recognized, and includes relatively non-toxic, inorganic and organic acid addition salts of compositions of the present invention, including without limitation, antineoplastic taxanes, excipients, other materials and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine; (trihydroxymethyl)aminoethane; and the like. See, for example, J. Pharm. Sci., 66:1-19 (1977).

A “patient,” “subject,” or “host” to be treated by the subject method may mean either a human or non-human animal, such as primates, mammals, and vertebrates.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

The term “preventing”, when used in relation to a condition, such as a local recurrence, a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount. Prevention of an infection includes, for example, reducing the number of diagnoses of the infection in a treated population versus an untreated control population, and/or delaying the onset of symptoms of the infection in a treated population versus an untreated control population.

“Radiosensitizer” is defined as a therapeutic agent that, upon administration in a therapeutically effective amount, promotes the treatment of one or more diseases or conditions that are treatable with electromagnetic radiation. In general, radiosensitizers are intended to be used in conjunction with electromagnetic radiation as part of a prophylactic or therapeutic treatment. Appropriate radiosensitizers to use in conjunction with treatment with the subject compositions will be known to those of skill in the art.

“Electromagnetic radiation” as used in this specification includes, but is not limited to, radiation having the wavelength of 10⁻²⁰ to 10 meters. Particular embodiments of electromagnetic radiation of the present invention employ the electromagnetic radiation of: gamma-radiation (10⁻²⁰ to 10⁻¹³ m), x-ray radiation (10⁻¹¹ to 10⁻⁹ m), ultraviolet light (10 nm to 400 nm), visible light (400 nm to 700 nm), infrared radiation (700 nm to 1.0 mm), and microwave radiation (1 mm to 30 cm).

The phrases “systemic administration,” “administered systemically,” “peripheral administration” and “administered peripherally” are art-recognized, and include the administration of a subject composition or other material at a site remote from the disease being treated. Administration of an agent directly into, onto or in the vicinity of a lesion of the disease being treated, even if the agent is subsequently distributed systemically, may be termed “local” or “topical” or “regional” administration, other than directly into the central nervous system, e.g., by subcutaneous administration, such that it enters the patient's system and, thus, is subject to metabolism and other like processes.

The terms “therapeutic agent”, “drug”, “medicament” and “bioactive substance” are art-recognized and include molecules and other agents that are biologically, physiologically, or pharmacologically active substances that act locally or systemically in a patient or subject to treat a disease or condition, such as prostate cancer, or a prostate tumor. Such agents may be acidic, basic, or salts; they may be neutral molecules, polar molecules, or molecular complexes capable of hydrogen bonding; they may be prodrugs in the form of ethers, esters, amides and the like that are biologically activated when administered into a patient or subject. Antineoplastic agents are exemplary therapeutic agents.

The phrase “therapeutically effective amount” is an art-recognized term. In certain embodiments, the term refers to an amount of an antineoplastic agent or other therapeutic agent (such as an antineoplastic taxane) that, when incorporated into a polymer of the present invention, produces some desired effect at a reasonable benefit/risk ratio applicable to any medical treatment. In certain embodiments, the term refers to that amount necessary or sufficient to eliminate, reduce or maintain (e.g., prevent the spread of) a tumor or other target of a particular therapeutic regimen. The effective amount may vary depending on such factors as the disease or condition being treated, the particular targeted constructs being administered, the size of the subject or the severity of the disease or condition. One of ordinary skill in the art may empirically determine the effective amount of a particular compound without necessitating undue experimentation.

In certain embodiments, a therapeutically effective amount of an antineoplastic agent, such as an antineoplastic taxane, for in vivo use will likely depend on a number of factors, including: the rate of release of the agent from the polymer matrix, which will depend in part on the chemical and physical characteristics of the polymer; the identity of the agent; the mode and method of administration; and any other materials incorporated in the polymer matrix in addition to the agent.

The term “ED₅₀” is art-recognized. In certain embodiments, ED₅₀ means the dose of a drug which produces 50% of its maximum response or effect, or alternatively, the dose which produces a pre-determined response in 50% of test subjects or preparations. The term “LD₅₀” is art-recognized. In certain embodiments, LD₅₀ means the dose of a drug which is lethal in 50% of test subjects. The term “therapeutic index” is an art-recognized term which refers to the therapeutic index of a drug, defined as LD₅₀/ED₅₀.

The terms “incorporated” and “encapsulated” are art-recognized when used in reference to an antineoplastic agent, (or other material) and a polymeric composition, such as a composition of the present invention. In certain embodiments, these terms include incorporating, formulating or otherwise including such agent into a composition which allows for sustained release of such agent in the desired application. The terms may contemplate any manner by which an antineoplastic agent or other material is incorporated into a polymer matrix, including for example: attached to a monomer of such polymer (by covalent or other binding interaction) and having such monomer be part of the polymerization to give a polymeric formulation, distributed throughout the polymeric matrix, appended to the surface of the polymeric matrix (by covalent or other binding interactions), encapsulated inside the polymeric matrix, etc. The term “co-incorporation” or “co-encapsulation” refers to the incorporation of an antineoplastic agent or other material and at least one other antineoplastic agent or other material in a subject composition.

More specifically, the physical form in which any antineoplastic agent or other material is encapsulated in polymers may vary with the particular embodiment. For example, an antineoplastic agent or other material may be first encapsulated in a microsphere and then combined with the polymer in such a way that at least a portion of the microsphere structure is maintained. Alternatively, an antineoplastic agent or other material may be sufficiently immiscible in the polymer of the invention that it is dispersed as small droplets, rather than being dissolved, in the polymer. Any form of encapsulation or incorporation is contemplated by the present invention, in so much as the sustained release of any encapsulated antineoplastic agent or other material determines whether the form of encapsulation is sufficiently acceptable for any particular use.

The term “biocompatible plasticizer” is art-recognized, and includes materials which are soluble or dispersible in the compositions of the present invention, which increase the flexibility of the polymer matrix, and which, in the amounts employed, are biocompatible. Suitable plasticizers are well known in the art and include those disclosed in U.S. Pat. Nos. 2,784,127 and 4,444,933. Specific plasticizers include, by way of example, acetyl tri-n-butyl citrate (c. 20 weight percent or less), acetyl trihexyl citrate (c. 20 weight percent or less), butyl benzyl phthalate, dibutyl phthalate, dioctylphthalate, n-butyryl tri-n-hexyl citrate, diethylene glycol dibenzoate (c. 20 weight percent or less) and the like.

“Small molecule” is an art-recognized term. In certain embodiments, this term refers to a molecule which has a molecular weight of less than about 2000 amu, or less than about 1000 amu, and even less than about 500 amu.

The term “aliphatic” is an art-recognized term and includes linear, branched, and cyclic alkanes, alkenes, or alkynes. In certain embodiments, aliphatic groups in the present invention are linear or branched and have from 1 to about 20 carbon atoms.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure.

Moreover, the term “alkyl” (or “lower alkyl”) includes both “unsubstituted alkyls” and “substituted alkyls”, the latter of which refers to alkyl moieties having substituents replacing a hydrogen on one or more carbons of the hydrocarbon backbone. Such substituents may include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain may themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls may be further substituted with alkyls, alkenyls, alkoxys, alkylthios, aminoalkyls, carbonyl-substituted alkyls, —CF₃, —CN, and the like.

The term “aralkyl” is art-recognized, and includes alkyl groups substituted with an aryl group (e.g., an aromatic or heteroaromatic group).

The terms “alkenyl” and “alkynyl” are art-recognized, and include unsaturated aliphatic groups analogous in length and possible substitution to the alkyls described above, but that contain at least one double or triple bond respectively.

Unless the number of carbons is otherwise specified, “lower alkyl” refers to an alkyl group, as defined above, but having from one to ten carbons, alternatively from one to about six carbon atoms in its backbone structure. Likewise, “lower alkenyl” and “lower alkynyl” have similar chain lengths.

The term “heteroatom” is art-recognized, and includes an atom of any element other than carbon or hydrogen. Illustrative heteroatoms include boron, nitrogen, oxygen, phosphorus, sulfur and selenium, and alternatively oxygen, nitrogen or sulfur.

The term “aryl” is art-recognized, and includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The terms ortho, meta and para are art-recognized and apply to 1,2-, 1,3- and 1,4-disubstituted benzenes, respectively. For example, the names 1,2-dimethylbenzene and ortho-dimethylbenzene are synonymous.

The terms “heterocyclyl” and “heterocyclic group” are art-recognized, and include 3- to about 10-membered ring structures, such as 3- to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” and “polycyclic group” are art-recognized, and include structures with two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms, e.g., three or more atoms are common to both rings, are termed “bridged” rings. Each of the rings of the polycycle may be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle” is art recognized and includes an aromatic or non-aromatic ring in which each atom of the ring is carbon. The flowing art-recognized terms have the following meanings: “nitro” means —NO₂; the term “halogen” designates —F, —Cl, —Br or —I; the term “sulfhydryl” means —SH; the term “hydroxyl” means —OH; and the term “sulfonyl” means —SO₂⁻.

The terms “amine” and “amino” are art-recognized and include both unsubstituted and substituted amines, e.g., a moiety that may be represented by the general formulas:

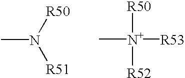

wherein R50, R51 and R52 each independently represent a hydrogen, an alkyl, an alkenyl, —(CH₂)ₘ-R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In certain embodiments, only one of R50 or R51 may be a carbonyl, e.g., R50, R51 and the nitrogen together do not form an imide. In other embodiments, R50 and R51 (and optionally R52) each independently represent a hydrogen, an alkyl, an alkenyl, or —(CH₂)ₘ-R61. Thus, the term “alkylamine” includes an amine group, as defined above, having a substituted or unsubstituted alkyl attached thereto, i.e., at least one of R50 and R51 is an alkyl group.

The term “acylamino” is art-recognized and includes a moiety that may be represented by the general formula:

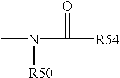

wherein R50 is as defined above, and R54 represents a hydrogen, an alkyl, an alkenyl or —(CH₂)ₘ-R61, where m and R61 are as defined above.

The term “amido” is art recognized as an amino-substituted carbonyl and includes a moiety that may be represented by the general formula:

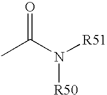

wherein R50 and R51 are as defined above. Certain embodiments of the amide in the present invention will not include imides which may be unstable.

The term “alkylthio” is art recognized and includes an alkyl group, as defined above, having a sulfur radical attached thereto. In certain embodiments, the “alkylthio” moiety is represented by one of —S-alkyl, —S-alkenyl, —S-alkynyl, and —S—(CH₂)ₘ-R61, wherein m and R61 are defined above. Representative alkylthio groups include methylthio, ethyl thio, and the like.

The term “carbonyl” is art recognized and includes such moieties as may be represented by the general formulas:

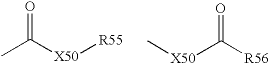

wherein X50 is a bond or represents an oxygen or a sulfur, and R55 represents a hydrogen, an alkyl, an alkenyl, —(CH₂)ₘ-R61 or a pharmaceutically acceptable salt, R56 represents a hydrogen, an alkyl, an alkenyl or —(CH₂)ₘ-R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiocarbonyl” group. Where X50 is a sulfur and R55 or R56 is not hydrogen, the formula represents a “thioester.” Where X50 is a sulfur and R55 is hydrogen, the formula represents a “thiocarboxylic acid.” Where X50 is a sulfur and R56 is hydrogen, the formula represents a “thioformate.” On the other hand, where X50 is a bond, and R55 is not hydrogen, the above formula represents a “ketone” group. Where X50 is a bond, and R55 is hydrogen, the above formula represents an “aldehyde” group.

The terms “alkoxyl” or “alkoxy” are art recognized and include an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ-R61, where m and R61 are described above.

The term “sulfonate” is art recognized and includes a moiety that may be represented by the general formula:

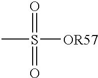

in which R57 is an electron pair, hydrogen, alkyl, cycloalkyl, or aryl.

The term “sulfate” is art recognized and includes a moiety that may be represented by the general formula:

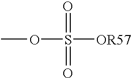

in which R57 is as defined above.

The term “sulfonamido” is art recognized and includes a moiety that may be represented by the general formula:

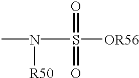

in which R50 and R56 are as defined above.

The term “sulfamoyl” is art-recognized and includes a moiety that may be represented by the general formula:

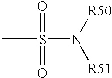

in which R50 and R51 are as defined above.

The term “sulfonyl” is art recognized and includes a moiety that may be represented by the general formula:

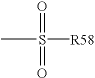

in which R58 is one of the following: hydrogen, alkyl, alkenyl, alkynyl, cycloalkyl, heterocyclyl, aryl or heteroaryl.

The term “sulfoxido” is art recognized and includes a moiety that may be represented by the general formula:

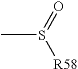

in which R58 is defined above.

The term “phosphoramidite” is art recognized and includes moieties represented by the general formulas:

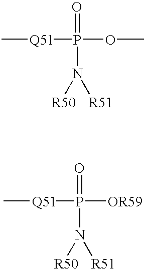

wherein Q51, R50, R51 and R59 are as defined above.

The term “phosphonamidite” is art recognized and includes moieties represented by the general formulas:

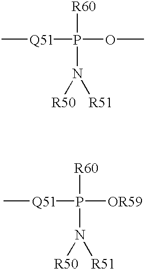

wherein Q51, R50, R51 and R59 are as defined above, and R60 represents a lower alkyl or an aryl.

Analogous substitutions may be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

The definition of each expression, e.g. alkyl, m, n, etc., when it occurs more than once in any structure, is intended to be independent of its definition elsewhere in the same structure unless otherwise indicated expressly or by the context.

The term “selenoalkyl” is art recognized and includes an alkyl group having a substituted seleno group attached thereto. Exemplary “selenoethers” which may be substituted on the alkyl are selected from one of —Se-alkyl, —Se-alkenyl, —Se-alkynyl, and —Se—(CH₂)ₘ-R61, m and R61 being defined above.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, and Ms are art recognized and represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

Certain monomeric subunits of the present invention may exist in particular geometric or stereoisomeric forms. In addition, polymers and other compositions of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

For purposes of this invention, the chemical elements are identified in accordance with the Periodic Table of the Elements, CAS version, Handbook of Chemistry and Physics, 67th Ed., 1986-87, inside cover. The term “hydrocarbon” is art recognized and includes all permissible compounds having at least one hydrogen and one carbon atom. For example, permissible hydrocarbons include acyclic and cyclic, branched and unbranched, carbocyclic and heterocyclic, aromatic and nonaromatic organic compounds that may be substituted or unsubstituted.

The phrase “protecting group” is art recognized and includes temporary substituents that protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed. Greene et al., Protective Groups in Organic Synthesis 2^{nd} ed., Wiley, N.Y., (1991).

The phrase “hydroxyl-protecting group” is art recognized and includes those groups intended to protect a hydroxyl group against undesirable reactions during synthetic procedures and includes, for example, benzyl or other suitable esters or ethers groups known in the art.

The term “electron-withdrawing group” is recognized in the art, and denotes the tendency of a substituent to attract valence electrons from neighboring atoms, i.e., the substituent is electronegative with respect to neighboring atoms. A quantification of the level of electron-withdrawing capability is given by the Hammett sigma (σ) constant. This well known constant is described in many references, for instance, March, Advanced Organic Chemistry 251-59, McGraw Hill Book Company, New York, (1977). The Hammett constant values are generally negative for electron donating groups (σ(P)=−0.66 for NH₂) and positive for electron withdrawing groups (σ(P)=0.78 for a nitro group), σ(P) indicating para substitution. Exemplary electron-withdrawing groups include nitro, acyl, formyl, sulfonyl, trifluoromethyl, cyano, chloride, and the like. Exemplary electron-donating groups include amino, methoxy, and the like.

Contemplated equivalents of the polymers, subunits and other compositions described above include such materials which otherwise correspond thereto, and which have the same general properties thereof (e.g., biocompatible, antineoplastic), wherein one or more simple variations of substituents are made which do not adversely affect the efficacy of such molecule to achieve its intended purpose. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here. 3. Exemplary Subject Compositions

Antineoplastic Agents and Other Therapeutic Molecules

A variety of antineoplastic agents are contemplated by the present invention.

Practitioners of the art will readily appreciate the circumstances under which various antineoplastic agents are appropriate for administration in the prostrate and/or for treatment of a prostrate cancer. For example, as described in the Exemplification section below, paclitaxel, an antineoplastic taxane, was used to treat prostrate cancers.

Non-limiting examples of antineoplastic agents include, in general, microtubule-stabilising agents (such as paclitaxel, docetaxel or their derivatives or analogs); alkylating agents; anti-metabolites; epidophyllotoxin; an antineoplastic enzyme; a topoisomerase inhibitor; procarbazine; mitoxantrone; platinum coordination complexes; biological response modifiers and growth inhibitors; and haematopoietic growth factors. Exemplary classes of antineoplastic agents include, for example, the anthracycline family of drugs, the vinca drugs, the mitomycins, the bleomycins, the cytotoxic nucleosides, the taxanes, the epothilones, discodermolide, the pteridine family of drugs, diynenes and the podophyllotoxins. Members of those classes include, for example, doxorubicin, carminomycin, daunorubicin, aminopterin, methotrexate, methopterin, dichloro-methotrexate, mitomycin C, porfiromycin, trastuzumab (Herceptin.TM.), 5-fluorouracil, 6-mercaptopurine, gemcitabine, cytosine arabinoside, podophyllotoxin or podo-phyllotoxin derivatives such as etoposide, etoposide phosphate or teniposide, melphalan, vinblastine, vincristine, leurosidine, vindesine, leurosine, paclitaxel and the like. Other useful antineoplastic agents include estramustine, cisplatin, carboplatin, cyclophosphamide, bleomycin, gemcitibine, ifosamide, melphalan, hexamethyl melamine, thiotepa, cytarabin, idatrexate, trimetrexate, dacarbazine, L-asparaginase, camptothecin, CPT-11, topotecan, pyridobenzoindole derivatives, interferons and interleukins.

Still other representative antineoplastic agents include: alkylating agents such as nitrogen mustards, for instance mechlorethamine, cyclophosphamide, melphatan and chlorambucil, alkyl sulptronates such as busulphan, nitrosoureas such as carmustine, lomusine, semustine and streptozocin, triazenes such as dacarbazine, antimetabolites such as folic acid analogues, for instance methotrexate, pyrimidine analogues such as fluorouracil and cytarabine, purine analogues such as mercaptopurine and thioguanine, natural products such as vinca alkaloids, for instance vinblastine, vincristine and vendesine, epipodophyllotoxins such as etoposide and teniposide, antibiotics such as dactinomycin, daunorubicin, doxorubicin, bleomycin, plicamycin and mitomycin, enzymes such as L-asparaginase, substituted ureas such as hydroxyurea, methylhydrazine derivatives such as procarbazine, adrenocorticoid suppressants such as mitotane and aminoglutethimide, hormones and antagonists such as adrenocorticosteroids such as prednisone, progestins such as hydroxyprogesterone caproate, methoxyprogesterone acetate and megestrol acetate, oestrogens such as diethylstilboestrol and ethinyloestradiol, antioestrogens such as tamoxifen, and androgens such as testosterone propionate and fluoxymesterone.

In certain embodiments, the antineoplastic agent is a member of the class of agents hereinafter defined as the antineoplastic taxanes, of which paclitaxel and docetaxel are two members. Paclitaxel and docetaxel share a common framework, and differ primarily in the substituents at two sites on this framework, shown as R1 and R2 in Formula I below:

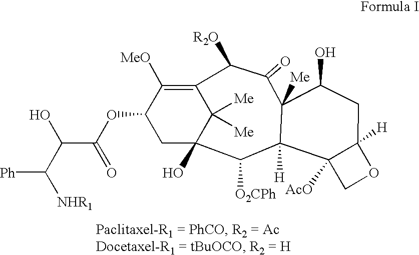

Thus, in one embodiment, a therapeutic composition of the invention comprises a compound of the above formula, wherein R1 is an acyl group or R1-N taken together comprise a carbamyl group (O—C(═O)—N), and R2 is H or an acyl group. In some embodiments, R1 comprises between 2 and 12 carbon atoms, or between 4 and 9 carbon atoms. In some embodiments, R2 is H or an acyl group having between 2 and 8 carbons, or between 2 and 4 carbons. In certain embodiments, the antineoplastic taxane is docetaxel or paclitaxel.

In another embodiment, a therapeutic composition of the present invention includes an antineoplastic agent having a structure of Formula II:

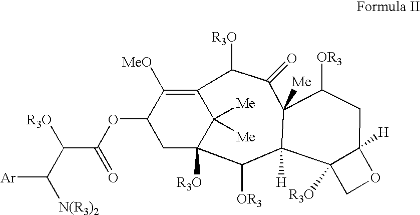

wherein, independently for each occurrence:

Ar represents a substituted or unsubstituted aryl or heteroaryl group; and

R3, each independently, represents H, alkyl, acyl, alkoxycarbonyl, aryloxycarbonyl, aminocarbonyl or sulfonyl.

In certain embodiments, at least one R3 is bound to nitrogen is H or alkyl. In certain embodiments, at least one R3 bound to nitrogen is acyl, alkoxycarbonyl, aryloxycarbonyl, aminocarbonyl, or sulfonyl. In certain embodiments, when R3 is bound to oxygen, R3 is selected from H, alkyl, acyl, aminocarbonyl, alkoxycarbonyl, or aryloxycarbonyl. In one embodiment, R3 is selected to be sterically similar to a corresponding substituent on paclitaxel or docetaxel, i.e., contains a number of carbon atoms within four of the number of carbon atoms in a similarly situated substituent of paclitaxel or docetaxel. For example, the benzoate ester of paclitaxel may be exchanged for a tosyl (p-toluenesulfonyl) ester, a cyclohexyl carbamate, or a tetrachlorobenzocyclopentanol carbonate, or a hydroxyl of docetaxel may be exchanged for an ethyl ether, a methylsulfonate ester, or a 2-hydroxyethyl carbamate.

In yet another embodiment, a therapeutic composition of the present invention includes an antineoplastic agent having a structure of Formula III:

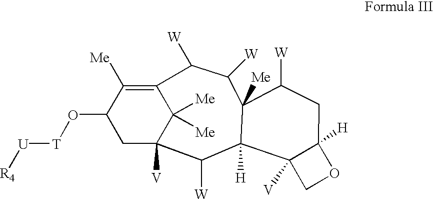

wherein, independently for each occurrence:

V, each independently, represents H, hydroxy, lower alkoxy, or a small ester (e.g., less than 4 carbons);

W, each independently, represents H, hydroxy, carbonyl, amino, alkoxy, sulfhydryl, alkylthio, ester, acylamino, carbamate, sulfonate, carbonate, or sulfoxide;

T represents —C(═O)—, —C(═S)—, —SO₂—, or —SO—;

U is absent or represents NH, S, or O; and

R4 represents a substituted aralkyl.

In certain embodiments, at least one occurrence of W or R4 includes a moiety, such as an oligopeptide or an oligosaccharide, that improves the bioavailability and/or solubility of the taxane. In certain embodiments, the therapeutic compound is formulated as a prodrug, e.g., at least one occurrence of W or R4 includes a moiety capable of being hydrolyzed and cleaved from the molecule under physiological conditions. The hydrolyzable moiety may improve the bioavailability and/or solubility of the taxane. The prodrug form of the therapeutic compound may itself be inactive, provided that after cleavage of the hydrolyzable moiety, the resulting compound is antineoplastic. In certain embodiments, at least one occurrence of W or R4 includes a bond to a polymer. The bond to the polymer may be hydrolyzable under physiologic conditions.

In certain embodiments, a therapeutic composition of the present invention includes an “antineoplastic taxane,” i.e., a compound which has a framework of Formula IV:

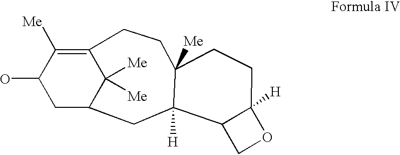

wherein, such framework bears sufficient substituents disposed at unspecified positions, as valence allows, such that the resulting compound has antineoplastic activity. In certain embodiments, such a compound is formed by chemically modifying paclitaxel or 10-deacetylbaccatin III, a naturally occurring compound which has the structure:

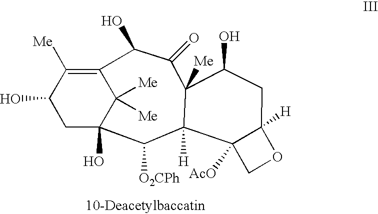

A variety of such antineoplastic derivatives are known in the art, and may be employed in the subject compositions and methods without departing from the spirit or scope of the present invention.

Still other antineoplastic agents will be known by those of skill in the art and may be encapsulated in the subject compositions without undue experimentation.

Polymers

A variety of polymers may be used in the subject invention. Both non-biodegradable and biodegradable polymers may be used in the subject invention. As discussed below, the choice of polymer will depend in part on a variety of physical and chemical characteristics of such polymer and the use to which such polymer may be put.

Representative natural polymers include proteins, such as zein, modified zein, casein, gelatin, gluten, serum albumin, or collagen, and polysaccharides, such as cellulose, dextrans, hyaluronic acid, and polymers of alginic acid.

Representative synthetic polymers include polyphosphazines, poly(vinyl alcohols), polyamides, polycarbonates, polyalkylenes, polyacrylamides, polyanhydrides, poly(phosphoesters), polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyphosphates and polyurethanes.

Synthetically modified natural polymers include alkyl celluloses, hydroxyalkyl celluloses, cellulose ethers, cellulose esters, and nitrocelluloses. Other like polymers of interest include, but are not limited to, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate and cellulose sulfate sodium salt.

Representative biodegradable polymers include polylactide, polyglycolide, polycaprolactone, polycarbonate, poly(phosphoesters), polyanhydride, polyorthoesters, and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins.

All of the subject polymers may be provided as copolymers or terpolymers. These polymers may be obtained from chemical suppliers or else synthesized from monomers obtained from these suppliers using standard techniques.

In addition to the listing of polymers above, polymers having phosphorus linkages may be used in the subject invention. Exemplary phosphorus linkages in such polymers include, without limitation, phosphonamidite, phosphoramidite, phosphorodiamidate, phosphomonoester, phosphodiester, phosphotriester, phosphonate, phosphonate ester, phosphorothioate, thiophosphate ester, phosphinate or phosphite. Certain of such polymers may be biodegradable, biocompatible or both.

The structure of certain of the foregoing polymers having phosphorus linkages may be identified as follows. The term “polymer having phosphorous-based linkages” is used herein to refer to polymers in which the following substructure is present at least a multiplicity of times in the backbone of such polymer:

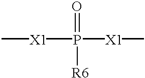

wherein, independently for each occurrence of such substructure:

X1, each independently, represents —O— or —N(R5)—;

R5 represents —H, aryl, alkenyl or alkyl; and

R6 is any non-interfering substituent,

wherein such substructure is responsible in part for biodegradability properties, if any, observed for such polymer in vitro or in vivo. In certain embodiments, R6 may represent an alkyl, aralkyl, alkoxy, alkylthio, or alkylamino group.

In certain embodiments, such a biodegradable polymer is non-naturally occurring, i.e., a man-made product with no natural source. In other embodiments, R6 is other than —OH or halogen, e.g., is alkyl, aralkyl, aryl, alkoxy, aralkyoxy or aryloxy. In still other embodiments, the two X1 moieties in such substructure are the same. For general guidance, when reference is made to the “polymer backbone chain” or the like of a polymer, with reference to the above structure, such polymer backbone chain comprises the motif [-X1-P-X1-]. In other polymers, the polymer backbone chain may vary as recognized by one of skill in the art.

By way of example, but not limitation, a number of representative polymers having phosphorus linkages are described in greater detail below. In certain embodiments, a polymer includes one or more monomeric units of Formula V:

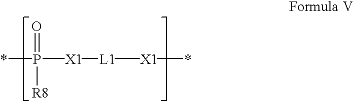

wherein, independently for each occurrence of such unit:

X1, each independently, represents —O— or —N(R7)—;

R7 represents —H, aryl, alkenyl or alkyl;

L1 is described below;

R8 represents, for example, —H, alkyl, —O-alkyl, —O-cycloalkyl, aryl, —O-aryl, heterocycle, —O-heterocycle, —N(R9)R10 and other examples presented below;

R9 and R10, each independently, represent a hydrogen, an alkyl, an alkenyl, —(CH2)m-R11, or R9 and R10, taken together with the N atom to which they are attached complete a heterocycle having from 4 to about 8 atoms in the ring structure;

m represents an integer in the range of 0-10, or 0-6; and

R11 represents —H, alkyl, aryl, cycloalkyl, cycloalkenyl, heterocycle or polycycle.

L1 may be any chemical moiety as long as it does not materially interfere with the polymerization, biocompatibility or biodegradation (or any combination of those three properties) of the polymer, wherein a “material interference” or “non-interfering substituent” is understood to mean: (i) for synthesis of the polymer by polymerization, an inability to prepare the subject polymer by methods known in the art or taught herein; (ii) for biocompatibility, a reduction in the biocompatibility of the subject polymer so as to make such polymer impracticable for in vivo use; and (iii) for biodegradation, a reduction in the biodegradation of the subject polymer so as to make such polymer impracticable for biodegradation.

In certain embodiments, L1 is an organic moiety, such as a divalent branched or straight chain or cyclic aliphatic group or divalent aryl group, with in certain embodiments, from 1 to about 20 carbon atoms. In certain embodiments, L1 represents a moiety between about 2 and 20 atoms selected from carbon, oxygen, sulfur, and nitrogen, wherein at least 60% of the atoms are carbon. In certain embodiments, LI may be an alkylene group, such as methylene, ethylene, 1,2-dimethylethylene, n-propylene, isopropylene, 2,2-dimethylpropylene, n-pentylene, n-hexylene, n-heptylene; an alkenylene group such as ethenylene, propenylene, 2-(3-propenyl)-dodecylene; and an alkynylene group such as ethynylene, proynylene, 1-(4-butynyl)-3-methyldecylene; and the like. Such unsaturated aliphatic groups may be used to cross-link certain embodiments of the present invention.

Further, L1 may be a cycloaliphatic group, such as cyclopentylene, 2-methylcyclopentylene, cyclohexylene, cyclohexylenedimethylene, cyclohexenylene and the like. L1 may also be a divalent aryl group, such as phenylene, benzylene, naphthalene, phenanthrenylene and the like. Further, L1 may be a divalent heterocyclic group, such as pyrrolylene, furanylene, thiophenylene, alkylyene-pyrrolylene-alkylene, pyridinylene, pyrimidinylene and the like.

Other examples of L1 may include any of the polymers listed above, including the biodegradable polymers listed above, and in particular polylactide, polyglycolide, polycaprolactone, polycarbonate, polyethylene terephthalate, polyanhydride and polyorthoester, and polymers of ethylene glycol, propylene glycol and the like. Embodiments containing such polymers for L1 may impart a variety of desired physical and chemical properties.

The foregoing, as with other moieties described herein, may be substituted with a non-interfering substituent, for example, a hydroxy-, halogen-, or nitrogen-substituted moiety.

R8 represents hydrogen, alkyl, cycloakyl, —O-alkyl, —O-cycloalkyl, aryl, —O-aryl, heterocycle, —O-heterocycle, or —N(R9)R10. Examples of possible alkyl R8 groups include methyl, ethyl, n-propyl, i-propyl, n-butyl, tert-butyl, —C₈H₁₇ and the like groups; and alkyl substituted with a non-interfering substituent, such as hydroxy, halogen, alkoxy or nitro; corresponding alkoxy groups.

When R8 is aryl or the corresponding aryloxy group, it typically contains from about 5 to about 14 carbon atoms, or about 5 to about 12 carbon atoms, and optionally, may contain one or more rings that are fused to each other. Examples of particularly suitable aromatic groups include phenyl, phenoxy, naphthyl, anthracenyl, phenanthrenyl and the like.

When R8 is heterocyclic or heterocycloxy, it typically contains from about 5 to about 14 ring atoms, alternatively from about 5 to about 12 ring atoms, and one or more heteroatoms. Examples of suitable heterocyclic groups include furan, thiophene, pyrrole, isopyrrole, 3-isopyrrole, pyrazole, 2-isoimidazole, 1,2,3-triazole, 1,2,4-triazole, oxazole, thiazole, isothiazole, 1,2,3-oxadiazole, 1,2,4-oxadiazole, 1,2,5-oxadiazole, 1,3,4-oxadiazole, 1,2,3,4-oxatriazole, 1,2,3,5-oxatriazole, 1,2,3-dioxazole, 1,2,4-dioxazole, 1,3,2-dioxazole, 1,3,4-dioxazole, 1,2,5-oxatriazole, 1,2-pyran, 1,4-pyran, 1,2-pyrone, 1,4-pyrone, 1,2-dioxin, 1,3-dioxin, pyridine, N-alkyl pyridinium, pyridazine, pyrimidine, pyrazine, 1,3,5-triazine, 1,2,4-triazine, 1,2,3-triazine, 1,2-oxazine, 1,3-oxazine, 1,4-oxazine, o-isoxazine, p-isoxazine, 1,2,5-oxathiazine, 1,2,6-oxathiazine, 1,4,2-oxadiazine, 1,3,5-oxadiazine, azepine, oxepin, thiepin, indene, isoindene, benzofuran, isobenzofuran, thionaphthene, isothionaphthene, indole, indolenine, 2-isobenzazole, isoindazole, indoxazine, benzoxazole, anthranil, 1,2-benzopyran, 1,2-benzopyrone, 1,4-benzopyrone, 2,1-benzopyrone, 2,3-benzopyrone, quinoline, isoquinoline, 12, -benzodiazine, 1,3-benzodiazine, naphthyridine, pyrido-[3,4-b]-pyridine, pyrido-[3,2-b]-pyridine, pyrido-[4,3-b]-pyridine, 1,3,2-benzoxazine, 1,4,2-benzoxazine, 2,3,1-benzoxazine, 3,1,4-benzoxazine, 1,2-benzisoxazine, 1,4-benzisoxazine, carbazole, xanthrene, acridine, purine, and the like. In certain embodiments, when R8 is heterocyclic or heterocycloxy, it is selected from the group consisting of furan, pyridine, N-alkylpyridine, 1,2,3- and 1,2,4-triazoles, indene, anthracene and purine rings.

In certain embodiments, R8 is an alkyl group, an alkoxy group, a phenyl group, a phenoxy group, a heterocycloxy group, or an ethoxy group.

In still other embodiments, R8, such as an alkyl, may be conjugated to a bioactive substance to form a pendant drug delivery system.

In certain embodiments, the number of monomeric units in Formula V and other subject formulas that make up the subject polymers ranges over a wide range, e.g., from about 2, 3, 4, 5 to 25,000 or more, but generally from about 50 to 5000, or 10,000. Alternatively, in other embodiments, the number of monomeric units may be about 10, 25, 50, 75, 100, 150, 200, 300 or 400.

In Formula V and other formulas herein, “*” represents other monomeric units of the subject polymer, which may be the same or different from the unit depicted in the formula in question, or a chain terminating group, by which the polymer terminates. Examples of such chain terminating groups include monofunctional alcohols and amines.

In another aspect, the polymeric compositions of the present invention include one or more recurring monomeric units represented in general Formula VI:

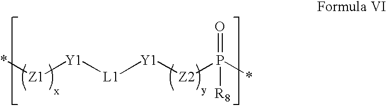

wherein Z1 and Z2, respectively, for each independent occurrence is:

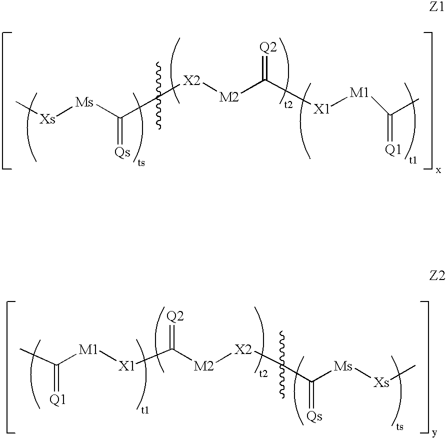

wherein, independently for each occurrence set forth above:

Q1, Q2 . . . Qs, each independently, represent O or N(R1);

X1, X2 . . . Xs, each independently, represent —O— or —N(R1);

the sum of t1, t2 . . . ts is an integer and at least one or more;

Y1 represents —O—, —S— or —N(R7)-;

x and y are each independently integers from 1 to about 1000 or more;

L1 and M1, M2 . . . Ms each independently, represent the moieties discussed below; and

the other moieties are as defined above.

M1, M2 . . . Ms (collectively, M) in Formula VI are each independently any chemical moiety that does not materially interfere with the polymerization, biocompatibility or biodegradation (or any combination of those three properties) of the subject polymer. For certain embodiments, M in the formula are each independently: (i) a branched or straight chain aliphatic or aryl group having from 1 to about 50 carbon atoms, or (ii) a branched or straight chain, oxa-, thia-, or aza-aliphatic group having from 1 to about 50 carbon atoms, both optionally substituted. In certain embodiments, the number of such carbon atoms does riot exceed 20. In other embodiments, M may be any divalent aliphatic moiety having from 1 to about 20 carbon atoms, including therein from 1 to about 7 carbon atoms.

M may include an aromatic or heteroaromatic moiety, optionally with non-interfering substituents. In certain embodiments, none of the atoms (usually but not always C) that form the cyclic ring that gives rise to the aromatic moiety are part of the polymer backbone chain.

Specifically, when M is a branched or straight chain aliphatic group having from 1 to about 20 carbon atoms, it may be, for example, an alkylene group such as methylene, ethylene, 1-methylethylene, 1,2-dimethylethylene, n-propylene, trimethylene, isopropylene, 2,2-dimethylpropylene, n-pentylene, n-hexylene, n-heptylene, n-octylene, n-nonylene, n-decylene, n-undecylene, n-dodecylene, and the like; an alkenylene group such as n-propenylene, 2-vinylpropylene, n-butenylene, 3-thexylbutylene, n-pentenylene, 4-(3-propenyl)hexylene, n-octenylene, 1-(4-butenyl)-3-methyldecylene, 2-(3-propenyl)dodecylene, hexadecenylene and the like; an alkynylene group, such as ethynylene, propynylene, 3-(2-ethynyl)pentylene, n-hexynylene, 2-(2-propynyl)decylene, and the like; or any alkylene, alkenylene or alkynylene group, including those listed above, substituted with a materially non-interfering substituent, for example, a hydroxy, halogen or nitrogen group, such as 2-chloro-n-decylene, 1-hydroxy-3-ethenylbutylene, 2-propyl-6-nitro-10-dodecynylene, and the like. Other M of the present invention include—(CH2)₃—, —(CH₂)₅— and —(CH₂)₂OCH₂—.

When M is a branched or straight chain oxaaliphatic group having from 1 to about 20 carbon atoms, it may be, for example, a divalent alkoxylene group, such as ethoxylene, 2-methylethoxylene, propoxylene, butoxylene, pentoxylene, dodecyloxylene, hexadecyloxylene, and the like. When M is a branched or straight chain oxaaliphatic group, it may have the formula —(CH₂)ₐ—O—(CH₂)_{b}— wherein each of a and b, independently, is about 1 to about 7.

When M is a branched or straight chain oxaaliphatic group having from 1 to about 20 carbon atoms, it may also be, for example, a dioxaalkylene group such as dioxymethylene, dioxyethylene, 1,3-dioxypropylene, 2-methoxy-1,3-dioxypropylene, 1,3-dioxy-2-methylpropylene, dioxy-n-pentylene, dioxy-n-octadecylene, methoxylene-methoxylene, ethoxylene-methoxylene, ethoxylene-ethoxylene, ethoxylene-1-propoxylene, butoxylene-n-propoxylene, pentadecyloxylene-methoxylene, and the like. When M is a branched or straight chain, dioxyaliphatic group, it may have the formula —(CH₂)ₐ—O—(CH₂)_{b}—O—(CH₂)_{c}—, wherein each of a, b, and c is independently from 1 to about 7.

When M is a branched or straight chain thiaaliphatic group, the group may be any of the preceding oxaaliphatic groups wherein the oxygen atoms are replaced by sulfur atoms.

When M is a branched or straight chain, aza-aliphatic group having from 1 to about 20 carbon atoms, it may be a divalent group such as —CH₂NH—, —(CH₂)₂N—, —CH₂(C₂H₅)N—, -n-C₄H₉NH—, -t-C₄H₉NH—, —CH₂(C₃H₇)N—, —C₂H₅(C₂H₅)N—, —CH₂(C₈H₁₇)N—, —CH₂NHCH₂—, —(CH₂)₂NCH₂—, —CH₂(C₂H₅)NCH₂CH₂—, -n-C₄H₉NHCH₂—, -t-C₄H₉NHCH₂CH₂—, —CH₂(C₃H₇)N(CH₂)₄—, —C₂H₅(C₂H₅)NCH₂—, —CH₂(C₈H₁₇)NCH₂CH₂—, and the like. When M is a branched or straight chain, amino-aliphatic group, it may have the formula —(CH₂)ₐNR1- or —(CH₂)ₐN(R1)(CH₂)_{b}— where R1 is —H, aryl, alkenyl or alkyl and each of a and b is independently from about 1 to about 7.

x and y of Formula VI each independently represent integers in the range of about 1 to about 1000, e.g., about 1, about 10, about 20, about 50, about 100, about 250, about 500, about 750, about 1000, etc.

For Formula VI, the average molar ratio of (x or y):L1, assuming ts is equal to one, may vary greatly, typically between about 75:1 and about 2:1. In certain embodiments, the average molar ratio of (x or y):L1, when ts is equal to one, is about 10:1 to about 4:1, or about 5:1. The molar ratio of x:y may also vary; typically, such ratio is about 1. Other possible embodiments may have ratios of 0.1, 0.25, 0.5, 0.75, 1.5, 2, 3, 4, 10 and the like.

A number of different polymer structures are contemplated by Formula VI. For example, in certain polymers exemplified by Formula VI, when the sum of t1, t2 . . . ts equals one for each of Z1 and Z2 and Q, M and X for each subunit ts are the same, then Formula VI becomes the following Formula VIa:

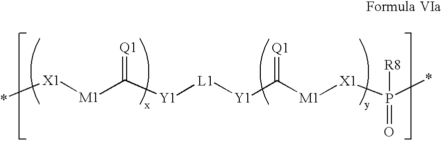

In certain embodiments of Formula VIa (and other subject formulas), x and y may be even integers.

The above Formula VI (and all of the subject formulae and polymers) encompass a variety of different polymer structures, including block copolymers, random copolymers, random terpolymers and segmented block copolymers and terpolymers. Additional structures for Z of subject monomeric units are set forth below, which exemplify in part the variety of structures contemplated by the present invention:

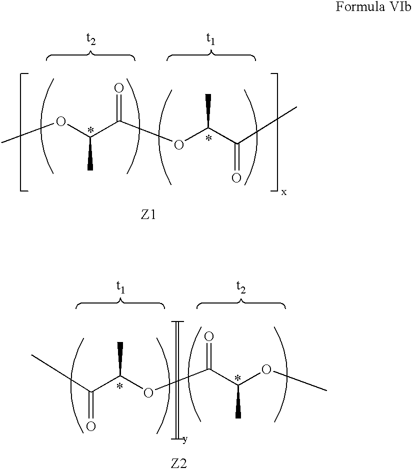

In Formula VIb (and other formulas described below), there may be more ts subunits depicted of the same molecular identity of those depicted in the formulas. For example, in Formula VIb, subunits t₁ and t₂ may be repeated in a sequence, e.g., alternating, in blocks (which may themselves repeat), or in any other pattern or random arrangement. Each subunit may repeat any number of times, and one subunit (e.g., t₁) may occur with substantially the same frequency, more often, or less often than another subunit (e.g., t₂), such that both subunits may be present in approximately the same amount, or in differing amounts, which may differ slightly or be highly disparate, e.g., one subunit is present nearly to the exclusion of the other. In certain embodiments, the chiral centers of each subunit may be the same or different and may be arranged in an orderly fashion or in a random sequence in each of Z1 and Z2.

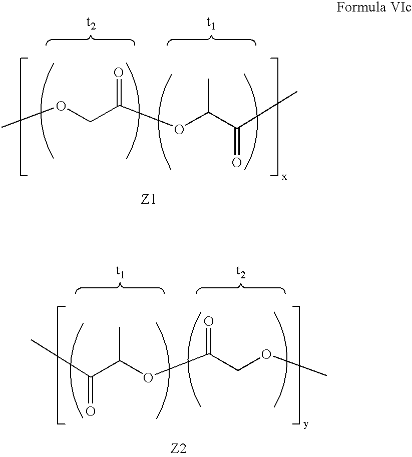

In certain embodiments of Formula VIc, the sum of the number of ts subunits in each of Z1 and Z2 is an even integer. As in other examples of Z1 and Z2, such as described above for Formula VIb, the ts subunits may be distributed randomly or in an ordered arrangement in each of Z1 or Z2.

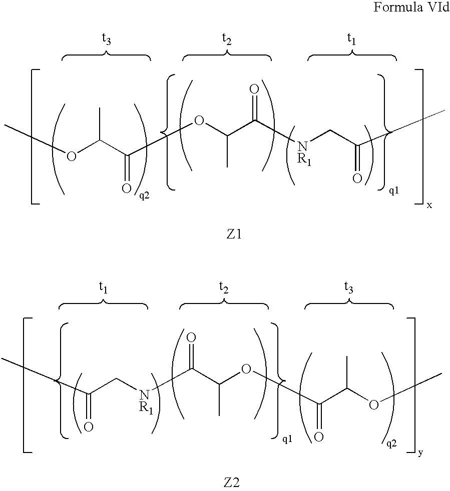

In Formula VId, the subunit q1 is comprised of two ts subunits, which may be repeated and arranged as described above for Formula VIb. In certain embodiments, q2 is an even integer, and in other embodiments, the subunits q1 and q2 may be distributed randomly or in an ordered pattern in each of Z1 and Z2. For example, subunits q₁ and q₂ may be repeated in a sequence, e.g., alternating, in blocks (which may themselves repeat), or in any other pattern or random arrangement. Each subunit may repeat any number of times, and one subunit (e.g., q₁) may occur with substantially the same frequency, more often, or less often than another subunit (e.g., q₂), such that both subunits may be present in approximately the same amount, or in differing amounts, which may differ slightly or be highly disparate, e.g., one subunit is present nearly to the exclusion of the other.

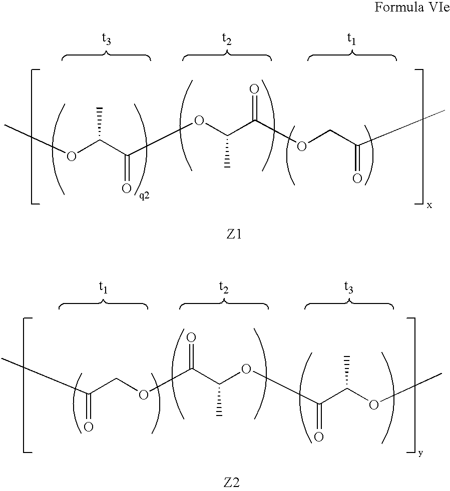

In certain embodiments of Formula VIe, the sum of the ts subunits for each of Z1 and Z2 is an even integer. In other embodiments, the each of the subunits t₁, t₂, and t₃ may be distributed randomly or in an ordered arrangement in each of Z1 and Z2. For example, in Formula VIe, subunits t₁, t₂, and t₃ may be repeated in a sequence, e.g., alternating, in blocks (which may themselves repeat), or in any other pattern or random arrangement. Each subunit may repeat any number of times, and one subunit (e.g., t₁) may occur with substantially the same frequency, more often, or less often than another subunit (e.g., t₃), such that the three subunits may be present in approximately the same amount, or in differing amounts, which may differ slightly or be highly disparate, e.g., two subunits are present nearly to the exclusion of the third.

In certain embodiments of Formula VI, in which Q, M and X for each subunit are the same, Q1 represents O, M represents a lower alkylene group, and X1 represents O or S. In one embodiment, X1 is O. For example, M may represent —CH(CH₃)— to result in a polymer of Formula VI having a structure represented in Formula VIf:

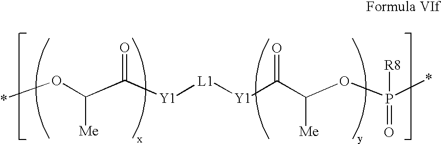

In certain embodiments of Formula VIf, as further described in the Exemplification below, L1 represents a lower alkylene chain, such as ethylene, propylene, etc. In certain embodiments, all Y1's represent O. In certain embodiments, R8 represents —O-lower alkyl, such as -OEt.

In certain embodiments of polymers depicted by Formula VI, the chirality of each subunit is identical, whereas in other embodiments, the chirality is different. By way of example but not limitation, in Formula VIb above, if the chiral centers of all of the subunits are D-enantiomers or L-enantiomers, then the monomeric unit is effectively equivalent to D-lactic acid or L-lactic acid, respectively, thereby giving rise to a region similar to poly(D-lactic acid) or poly(L-lactic acid), respectively. Conversely, if the two subunits in Formula VIb are comprised of alternating D- and L-enantiomers (e.g., one unit of D-enantiomer, one unit of L-enantiomer, etc.), then the resulting polymeric region is analogous to poly(meso-lactic acid) (i.e., a polymer formed by polymerization of meso-lactide).

Finally, in certain embodiments of the monomeric units set forth in Formula VI, in which the entire polymer may or may not be composed of such units, the following moieties for Y1, L1, R8, Qs, Xs and Ms may be used (with a variety of different x and y being possible):

| Abbreviation | All Y1's | L1 | R8 |
|---|---|---|---|
| L-PL(EG)EOP | O | —CH₂CH₂— | —OCH₂CH₃ |
| L-PL(EG)HOP | O | —CH₂CH₂— | —O(CH₂)₅CH₃ |
| D,L-PL(EG)EOP* | O | —CH₂CH₂— | —OCH₂CH₃ |
| D,L-PL(PG)EOP* | O | —CH₂(CH₃)CH₂— | —OCH₂CH₃ |
| D-PL(PG)EOP | O | —CH₂(CH₃)CH₂— | —OCH₂CH₃ |
| L-PL(PG)EOP | O | —CH₂(CH₃)CH₂— | —OCH₂CH₃ |
| D,L-PL(HD)EOP* | O | 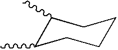 | —OCH₂CH₃ |
| D,L-PL(PG)HOP* | O | —CH₂(CH₃)CH₂— | —O(CH₂)₅CH₃ |
| D,L-PL(PG)EP* | O | —CH₂(CH₃)CH₂— | —CH₂CH₃ |

| Abbreviation | All Qs | All Xs | M1 | M2 |
|---|---|---|---|---|
| L-PL(EG)EOP | O | O | —CH(CH₃)— (L) | N/A |
| L-PL(EG)HOP | O | O | —CH(CH₃)— (L) | N/A |
| D,L-PL(EG)EOP* | O | O | —CH(CH₃)— (L or D) | —CH(CH₃)— (D or L) |
| D,L-PL(PG)EOP* | O | O | —CH(CH₃)— (L or D) | —CH(CH₃)— (D or L) |
| D-PL(PG)EOP | O | O | —CH(CH₃)— (D) | N/A |
| L-PL(PG)EOP | O | O | —CH(CH₃)— (L) | N/A |
| D,L-PL(HD)EOP* | O | O | —CH(CH₃)— (L or D) | —CH(CH₃)— (L or D) |
| D,L-PL(PG)HOP* | O | O | —CH(CH₃)— (L or D) | —CH(CH₃)— (L or D) |
| D,L-PL(PG)EP* | O | O | —CH(CH₃)— (L or D) | —CH(CH₃)— (L or D) |

*For D,L-PL(EG)EOP, D,L-PL(PG)EOP, D,L-PL(HD)EOP, D,L-PL(PG)HOP, and D,L-PL(PG)EP, if the chiral carbon of M1 has configuration L, then M2 will have configuration D, and vice-versa. The order of the chiral centers in each subunit M1 and M2 for each Z1 and Z2 will be in random order.

In addition to the particular chiral version of the subject polymers described in the above table, polymers in which the chirality of MS varies in each subunit M in the subject polymers are also possible. For instance, referring to D,L-PL(EG)EOP by example, a random order of D and L, in varying amounts, are possible for this polymer. In contrast, the table sets forth one such example in which a D and L chiral M are always adjacent, in equal amounts, but that need not always be the case.

In another embodiment of the present invention, the polymeric compositions of the present invention include one or more recurring monomeric units represented in general Formula VII:

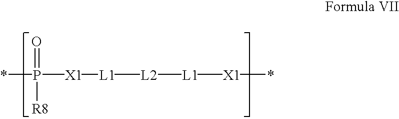

wherein, independently for each occurrence:

L2 is a divalent organic group as described in greater detail below; and

the other moieties are as defined as above.

In Formula VII, L2 may be a divalent, branched or straight chain aliphatic group, a cycloaliphatic group, or a group of the formula:

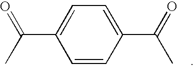

Specific examples of particular divalent, branched or straight chain aliphatic groups include an alkylene group with 1 to 7 carbon atoms, such as 2-methylpropylene or ethylene. Specific examples of cycloaliphatic groups include cycloalkylene groups, such as cyclopentylene, 2-methylcyclopentylene, cyclohexylene and 2-chloro-cyclohexylene; cycloalkenylene groups, such as cyclohexenylene; and cycloalkylene groups having fused or bridged additional ring structures, such as tetralinylene, decalinylene and norpinanylene; or the like.

In certain embodiments of the monomeric units set forth in Formula VII, in which the entire polymer may or may not be composed of such units, the following moieties for X1, L1 and R8 may be used:

| Abbreviation | All X1 | All L1 | L2 | R8 |
|---|---|---|---|---|
| P(trans-CHDM/HOP) | O | —CH2— | 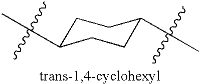 | —O(CH2)5CH3 |
| P(cis- and trans-CHDM/HOP) | O | —CH2— | 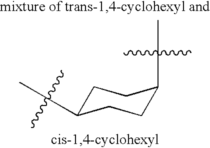 | —O(CH2)5CH3 |
| P(trans-CHDM/BOP) | O | —CH2— | trans-1,4-cyclohexyl | —O(CH2)3CH3 |
| P(trans-CHDM/EOP) | O | —CH2— | trans-1,4-cyclohexyl | —OCH2CH3 |

In another embodiment of the present invention, the polymeric compositions of the present invention include one or more recurring monomeric units represented in general Formula VIII:

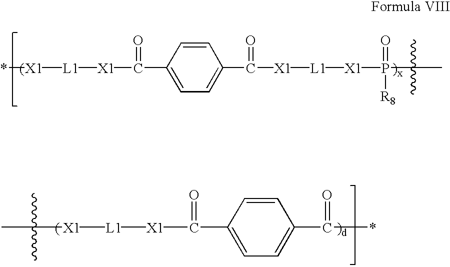

wherein, independently for each occurrence, d is equal to one or more, and optionally two, x is equal to or greater than one, and all of the other moieties are as defined above. In certain embodiments of Formula VIII, each of L1 independently may be an alkylene group, a cycloaliphatic group, a phenylene group or a divalent group of the formula:

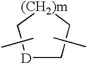

wherein D is O, N or S and m is 0 to 3. Alternatively, L1 is a branched or straight chain alkylyene group having from 1 to 7 carbon atoms, such as a methylene, ethylene, n-propylene, 2-methylpropylene, 2,2′-dimethylpropylene group and the like.

In certain embodiments of the monomeric units set forth in Formula VIII, in which the entire polymer may or may not be composed of such units, the following moieties for X1, L1 and R8 may be used (with a variety of different x possible for each example and in one embodiment, with d equal to two):

| Abbreviation | All X1 | All L1 | R8 |
|---|---|---|---|
| P(BHET-EOP/TC) | O | —CH2CH2— | —OCH2CH3 |
| P(BHDPT-EOP/TC) | O | —CH2CH(CH3)2CH2— | —OCH2CH3 |
| P(BHDPT-HOP/TC) | O | —CH2CH(CH3)2CH2— | —OC6H13 |
| P(BHPT-EOP/TC) | O | —CH2CH2CH2— | —OCH2CH3 |
| P(BHMPT-EOP/ | O | CH2CH2(CH3)CH2— | —OCH2CH3 |
| TC) | | | |

In Formula VIII, the aryl groups represented therein may be substituted with a non-interfering substituent, for example, a hydroxy-, halogen-, or nitrogen-substituted moiety.

Other phosphorus containing polymers which may be adapted for use in the subject invention, and methods of making the same, are described in the art, including those described in U.S. Pat. Nos. 5,256,765, 5,194,581, 6,166,173, 6,153,212, 6,322,797, 6,403,675, and 6,419,709; and PCT publications WO 98/44020, WO 98/44021, and WO 98/48859. For all of the above-identified groups, non-interfering substituents may also be present.

In certain embodiments, the polymers are comprised almost entirely, if not entirely, of the same subunit. Alternatively, in other embodiments, the polymers may be copolymers, in which different subunits and/or other monomeric units are incorporated into the polymer. In certain instances, the polymers are random copolymers, in which the different subunits and/or other monomeric units are distributed randomly throughout the polymer chain. For example, the polymer having units of Formula V may consist of effectively only one type of such subunit, or alternatively two or more types of such subunits. In addition, the polymer may contain monomeric units other than those subunits represented by Formula V.

In other embodiments, the different types of monomeric units, be they one or more subunits depicted by the subject formulas or other monomeric units, are distributed randomly throughout the chain. In part, the term “random” is intended to refer to the situation in which the particular distribution or incorporation of monomeric units in a polymer that has more than one type of monomeric units is not directed or controlled directly by the synthetic protocol, but instead results from features inherent to the polymer system, such as the reactivity, amounts of subunits and other characteristics of the synthetic reaction or other methods of manufacture, processing or treatment.

In certain embodiments, the subject polymers may be cross-linked. For example, substituents of the polymeric chain, may be selected to permit additional inter-chain cross-linking by covalent or electrostatic (including hydrogen-binding or the formation of salt bridges), e.g., by the use of a organic residue appropriately substituted.

The ratio of different subunits in any polymer as described above may vary. For example, in certain embodiments, polymers may be composed almost entirely, if not entirely, of a single monomeric element, such as a subunit depicted in Formula V. Alternatively, in other instances, the polymers are effectively composed of two different subunits, in which the percentage of each subunit may vary from less than 1:99 to more than 99:1, or alternatively 10:90, 15:85, 25:75, 40:60, 50:50, 60:40, 75:25, 85:15, 90:10 or the like. For example, in some instances, a polymer may be composed of two different subunits that may be both represented by the generic Formula V, but which differ in their chemical identity. In certain embodiments, the polymers may have just a few percent, or even less (for example, about 5, 2.5, 1, 0.5, 0.1%) of the subunits having phosphorous-based linkages. In other embodiments, in which three or more different monomeric units are present, the present invention contemplates a range of mixtures like those taught for the two-component systems.

In certain embodiments, the polymeric chains of the subject compositions, e.g., which include repetitive elements shown in any of the subject formulas, have molecular weights ranging from about 2000 or less to about 1,000,000 or more daltons, or alternatively about 10,000, 20,000, 30,000, 40,000, or 50,000 daltons, more particularly at least about 100,000 daltons, and even more specifically at least about 250,000 daltons or even at least 500,000 daltons. Number-average molecular weight (Mn) may also vary widely, but generally fall in the range of about 1,000 to about 200,000 daltons, or from about 1,000 to about 100,000 daltons or even from about 1,000 to about 50,000 daltons. In one embodiment, Mn varies between about 8,000 and 45,000 daltons. Within a given sample of a subject polymer, a wide range of molecular weights may be present. For example, molecules within the sample may have molecular weights which differ by a factor of 2, 5, 10, 20, 50, 100, or more, or which differ from the average molecular weight by a factor of 2, 5, 10, 20, 50, 100, or more.

One method to determine molecular weight is by gel permeation chromatography (“GPC”), e.g., mixed bed columns, CH₂Cl₂ solvent, light scattering detector, and off-line dn/dc. Other methods are known in the art.

In certain embodiments, the intrinsic viscosities of the polymers generally vary from about 0.01 to about 2.0 dL/g in chloroform at 40° C., alternatively from about 0.01 to about 1.0 dL/g and, occasionally, from about 0.01 to about 0.5 dL/g.

The glass transition temperature (Tg) of the subject polymers may vary widely, and depend on a variety of factors, such as the degree of branching in the polymer components, the relative proportion of phosphorous-containing monomer used to make the polymer, and the like. When the article of the invention is a rigid solid, the Tg is often within the range of from about −10° C. to about 80° C., particularly between about 0 and 50° C. and, even more particularly between about 25° C. to about 35° C. In other embodiments, the Tg may be low enough to keep the composition of the invention flowable at body temperature. Then, the glass transition temperature of the polymer used in the invention is usually about 0 to about 37° C., or alternatively from about 0 to about 25° C.

In certain embodiments, substituents of the phosphorus atom, such as R8 in the above formulas, and other components of the subject polymers may permit additional inter-chain cross-linking by covalent or electrostatic interactions (including, for example, hydrogen-binding or the formation of salt bridges) by having a side chain of either of them appropriately substituted as discussed in greater detail below.

In other embodiments, the polymer composition of the invention may be a flexible or flowable material. When the polymer used is itself flowable, the polymer composition of the invention, even when viscous, need not include a biocompatible solvent to be flowable, although trace or residual amounts of biocompatible solvents may still be present.

In certain embodiments, a fluid polymer may be especially suitable for the treatment of prostate cancers. A fluid material may be adapted for injection or instillation into a tissue mass or into an actual or potential space. Certain types of fluid polymers may be termed flowable. A flowable material, often capable of assuming the shape of the contours of an irregular space, may be delivered to a portion of an actual or potential space to flow therefrom into a larger portion of the space. In this way, the flowable material may come to coat an entire post-operative surgical site after being inserted through an edge of an incision or after being instilled through a drain or catheter left in the surgical bed. Alternatively, if the flowable material is inserted under pressure through a device such as a needle or a catheter, it may perform hydrodissection, thus opening up a potential space and simultaneously coating the space with polymer. Such potential spaces suitable for hydrodissection may be found in various identifiable anatomic areas in the pelvis where prostate cancers may require treatment. For example, in performing endoscopic lymph node biopsy or lymphadenectomy, hydrodissection, instrument dissection or their art-recognized equivalents may be performed to allow the flowable material to coat or to contact certain of the lymph nodes or their excision beds. As is known in the art, the retroperitoneum is suitable for such techniques and may permit the introduction of flowable materials according to the present invention into the retroperitoneal space to occupy the anatomic areas where the reelvant lymph nodes reside. A flowable polymer may be particularly adapted for instillation through a needle, catheter or other delivery device such as an endoscope, since its flowable characteristics allow it to reach surfaces that extend beyond the immediate reach of the delivery device. A flowable polymer in a highly fluid state may be suitable for injection through needles or catheters into tissue masses, such as tumors or margins of resection sites. Physical properties of polymers may be adjusted to achieve a desirable state of fluidity or flowability by modification of their chemical components and crosslinking, using methods familiar to practitioners of ordinary skill in the art.

A flexible polymer may be used in the fabrication of a solid article. Flexibility involves having the capacity to be repeatedly bent and restored to its original shape. Solid articles made from flexible polymers are adapted for placement in anatomic areas where they will encounter the motion of adjacent organs or body walls. Certain areas of motion are familiar to practitioners dealing with prostate tumors. A flexible solid article can thus be sufficiently deformed by those moving tissues that it does not cause tissue damage. Flexibility is particularly advantageous where a solid article might be dislodged from its original position and thereby encounter an unanticipated moving structure; flexibility may allow the solid article to bend out of the way of the moving structure instead of injuring it. Such a flexible article might be suitable for covering pulsatile vessels in the pelvis that may be in proximity to a locally advanced prostate cancer, or for being juxtaposed to a hollow viscus such as bowel or bladder where erosion through the wall might produce significant morbidity. Similarly, a flexible solid article may be used to protect nerves exposed during a dissection in proximity to the prostate cancer, wherein the flexibility of the solid article may permit it to bend or deform when encountering motion rather than eroding into or damaging the nerve. Use of a solid article according to the present invention in the aforesaid ways may allow less extensive dissections to be carried out with surgical preservation and antineoplastic protection of structures important to function. Solid articles may be configured as three-dimensional structures suitable for implantation in specific anatomic areas. For example, a solid article may be formed to be implantable within a bony metastasis of prostate cancer in an appropriate patient, said solid article being adapted in certain embodiments for preserving or supplementing bony strength that has been eroded by the metastasis and furthermore carrying an antineoplastic taxane. Solid articles may be formed as films, meshes, sheets, tubes, or any other shape appropriate to the dimensions and functional requirements of the particular anatomic area. Physical properties of polymers may be adjusted to attain a desirable degree of flexibility by modification of the chemical components and crosslinking thereof, using methods familiar to practitioners of ordinary skill in the art.

While it is possible that the biocompatible polymer or the biologically active agent may be dissolved in a small quantity of a solvent that is non-toxic to more efficiently produce an amorphous, monolithic distribution or a fine dispersion of the biologically active agent in the flexible or flowable composition, it is an advantage of the invention that, in an embodiment, no solvent is needed to form a flowable composition. Moreover, the use of solvents may be avoided because, once a polymer composition containing solvent is placed totally or partially within the body, the solvent dissipates or diffuses away from the polymer and must be processed and eliminated by the body, placing an extra burden on the body's clearance ability at a time when the illness (and/or other treatments for the illness) may have already deleteriously affected it.

However, when a solvent is used to facilitate mixing or to maintain the flowability of the polymer composition of the invention, it should be non-toxic, otherwise biocompatible, and should be used in relatively small amounts. Solvents that are toxic clearly should not be used in any material to be placed even partially within a living body. Such a solvent also must not cause substantial tissue irritation or necrosis at the site of administration.

Examples of suitable biocompatible solvents, when used, include N-methyl-2-pyrrolidone, 2-pyrrolidone, ethanol, propylene glycol, acetone, methyl acetate, ethyl acetate, methyl ethyl ketone, dimethylformamide, dimethyl sulfoxide, tetrahydrofuran, caprolactam, dimethyl-sulfoxide, oleic acid, or 1-dodecylazacycloheptan-2-one. Solvents may include N-methyl-2-pyrrolidone, 2-pyrrolidone, dimethyl sulfoxide, and acetone because of their solvating ability and their biocompatibility.

The microspheres may be manufactured by incorporating the drug into the polymer matrix by either dissolving or suspending the drug into polymer solution and the mixture will be subsequently dried by techniques familiar to those skill in the arts to form microspheres. These techniques include but not limited to spray drying, coating, various emulsion methods and supercritical fluid processing. The microspheres may be mixed with a pharmaceutically acceptable diluent prior to the administration for injection. They may also be directly applied to the desired site, such as a surgical wound or cavity, by various delivery systems including pouring and spraying. The microspheres may also be mixed with pharmaceutically acceptable ingredients to create ointment or cream for topical applications.

Therapeutic Compositions

The antineoplastic agents of the present invention are used in amounts that are therapeutically effective, which varies widely depending largely on the particular antineoplastic agent being used. The amount of antineoplastic agent incorporated into the composition also depends upon the desired release profile, the concentration of the agent required for a biological effect, and the length of time that the biologically active substance has to be released for treatment. In certain embodiments, the biologically active substance may be blended with the polymer matrix of the invention at different loading levels, in one embodiment, at room temperature and without the need for an organic solvent. In other embodiments, the compositions of the present invention may be formulated as microspheres.

There is no critical upper limit on the amount of antineoplastic agent incorporated except for that of an acceptable solution or dispersion viscosity to maintain the physical characteristics desired for the composition. The lower limit of the antineoplastic agent incorporated into the polymers system is dependent upon the activity of the drug and the length of time needed for treatment. Thus, the amount of the antineoplastic agent should not be so small that it fails to produce the desired physiological effect, nor so large that the antineoplastic agent is released in an uncontrollable manner. Typically, within these limits, amounts of the antineoplastic agent from about 1% up to about 60% may be incorporated into the present delivery systems. However, lesser amounts may be used to achieve efficacious levels of treatment for antineoplastic agent that are particularly potent.

In addition, the polymer compositions of the invention may comprise blends of the polymer of the invention with other biocompatible polymers or copolymers, so long as the additional polymers or copolymers do not interfere undesirably with the biocompatible, biodegradable and/or mechanical characteristics of the composition. Blends of the polymer of the invention with such other polymers may offer even greater flexibility in designing the precise release profile desired for targeted drug delivery or the precise rate of biodegradability desired. Examples of such additional biocompatible polymers include other poly(phosphoesters), poly(carbonates), poly(esters), poly(orthoesters), poly(amides), poly(urethanes), poly(imino-carbonates), and poly(anhydrides).

Pharmaceutically acceptable polymeric carriers may also comprise a wide range of additional materials. Without being limited thereto, such materials may include diluents, binders and adhesives, lubricants, disintegrants, colorants, bulking agents, flavorings, sweeteners, and miscellaneous materials such as buffers and adsorbents, in order to prepare a particular medicated composition, with the condition that none of these additional materials will interfere with the intended purpose of the subject composition.

For delivery of an antineoplastic agent or some other biologically active substance, the agent or substance is added to the polymer composition. A variety of methods are known in the art for encapsulating a biologically active substance in a polymer. For example, the agent or substance may be dissolved to form a homogeneous solution of reasonably constant concentration in the polymer composition, or it may be dispersed to form a suspension or dispersion within the polymer composition at a desired level of “loading” (grams of biologically active substance per grams of total composition including the biologically active substance, usually expressed as a percentage).

In part, a polymer composition of the present invention useful in the treatment of prostate cancer includes both: (a) an antineoplastic agent, and (b) a biocompatible and optionally biodegradable polymer, such as one having the recurring monomeric units shown in one of the foregoing formulas, or any other biocompatible polymer mentioned above or known in the art.

In certain embodiments in which the subject composition will be used to treat prostate cancers, the antineoplastic agent is an antineoplastic taxane, such as paclitaxel, docetaxel, and analogs thereof, or another antineoplastic taxane. In still other embodiments, the subject compositions may encapsulate more than one antineoplastic agent for treatment of prostate cancer.

Any additional therapeutic substance in a subject composition may vary widely with the purpose for the composition. The term therapeutic agent includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

Plasticizers and stabilizing agents known in the art may be incorporated in polymers of the present invention. In certain embodiments, additives such as plasticizers and stabilizing agents are selected for their biocompatibility.

A composition of this invention may further contain one or more adjuvant substances, such as fillers, thickening agents or the like. In other embodiments, materials that serve as adjuvants may be associated with the polymer matrix. Such additional materials may affect the characteristics of the polymer matrix that results. For example, fillers, such as bovine serum albumin (BSA) or mouse serum albumin (MSA), may be associated with the polymer matrix. In certain embodiments, the amount of filler may range from about 0.1 to about 50% or more by weight of the polymer matrix, or about 2.5, 5, 10, 25, 40 percent. Incorporation of such fillers may affect the biodegradation of the polymeric material and/or the sustained release rate of any encapsulated substance. Other fillers known to those of skill in the art, such as carbohydrates, sugars, starches, saccharides, celluoses and polysaccharides, including mannitose and sucrose, may be used in certain embodiments in the present invention.

In other embodiments, spheronization enhancers facilitate the production of subject polymeric matrices that are generally spherical in shape. Substances such as zein, microcrystalline cellulose or microcrystalline cellulose co-processed with sodium carboxymethyl cellulose may confer plasticity to the subject compositions as well as implant strength and integrity. In particular embodiments, during spheronization, extrudates that are rigid, but not plastic, result in the formation of dumbbell shaped implants and/or a high proportion of fines, and extrudates that are plastic, but not rigid, tend to agglomerate and form excessively large implants. In such embodiments, a balance between rigidity and plasticity is desirable. The percent of spheronization enhancer in a formulation depends on the other excipient characteristics and is typically in the range of 10-90% (w/w).

Buffers, acids and bases may be incorporated in the subject compositions to adjust their pH. Agents to increase the diffusion distance of agents released from the polymer matrix may also be included.

Disintegrants are substances which, in the presence of liquid, promote the disruption of the subject compositions. Disintegrants are most often used in implants, in which the function of the disintegrant is to counteract or neutralize the effect of any binding materials used in the subject formulation. In general, the mechanism of disintegration involves moisture absorption and swelling by an insoluble material. Examples of disintegrants include croscarmellose sodium and crospovidone that, in certain embodiments, may be incorporated into the polymeric matrices in the range of about 1-20% of total matrix weight. In other cases, soluble fillers such as sugars (mannitol and lactose) may also be added to facilitate disintegration of the subject composition upon use.

Other materials may be used to advantage to control the desired release rate of a antineoplastic agent for a particular treatment protocol. For example, if the sustained release is too slow for a particular application, a pore-forming agent may be added to generate additional pores in the matrix. Any biocompatible water-soluble material may be used as the pore-forming agent. They may be capable of dissolving, diffusing or dispersing out of the formed polymer system whereupon pores and microporous channels are generated in the system. The amount of pore-forming agent (and size of dispersed particles of such pore-forming agent, if appropriate) within the composition should affect the size and number of the pores in the polymer system.

Pore-forming agents include any pharmaceutically acceptable organic or inorganic substance that is substantially miscible in water and body fluids and will dissipate from the forming and formed matrix into aqueous medium or body fluids or water-immiscible substances that rapidly degrade to water-soluble substances. Suitable pore-forming agents include, for example, sugars such as sucrose and dextrose, salts such as sodium chloride and sodium carbonate, and polymers such as hydroxylpropylcellulose, carboxymethylcellulose, polyethylene glycol, and polyvinylpyrrolidone. The size and extent of the pores may be varied over a wide range by changing the molecular weight and percentage of pore-forming agent incorporated into the polymer system.

The charge, lipophilicity or hydrophilicity of any subject polymeric matrix may be modified by attaching in some fashion an appropriate compound to the surface of the matrix. For example, surfactants may be used to enhance wettability of poorly soluble or hydrophobic compositions. Examples of suitable surfactants include dextran, polysorbates and sodium lauryl sulfate. In general, surfactants are used in low concentrations, generally less than about 5%.

Binders are adhesive materials that may be incorporated in polymeric formulations to bind and maintain matrix integrity. Binders may be added as dry powder or as solution. Sugars and natural and synthetic polymers may act as binders. Materials added specifically as binders are generally included in the range of about 0.5%-15% w/w of the matrix formulation. Certain materials, such as microcrystalline cellulose, also used as a spheronization enhancer, also have additional binding properties.

Various coatings may be applied to modify the properties of the matrices. Three exemplary types of coatings are seal, gloss and enteric coatings. Other types of coatings having various dissolution or erosion properties may be used to further modify subject matrices behavior, and such coatings are readily known to one of ordinary skill in the art.

The seal coat may prevent excess moisture uptake by the matrices during the application of aqueous based enteric coatings. The gloss coat generally improves the handling of the finished matrices. Water-soluble materials such as hydroxypropyl cellulose may be used to seal coat and gloss coat implants. The seal coat and gloss coat are generally sprayed onto the matrices until an increase in weight between about 0.5% and about 5%, often about 1% for a seal coat and about 3% for a gloss coat, has been obtained.

Enteric coatings consist of polymers which are insoluble in the low pH (less than 3.0) of the stomach, but are soluble in the elevated pH (greater than 4.0) of the small intestine. Polymers such as EUDRAGIT, RohmTech, Inc., Malden, Mass., and AQUATERIC, FMC Corp., Philadelphia, Penn., may be used and are layered as thin membranes onto the implants from aqueous solution or suspension or by a spray drying method. The enteric coat is generally sprayed to a weight increase of about one to about 30%, even about 10 to about 15% and may contain coating adjuvants such as plasticizers, surfactants, separating agents that reduce the tackiness of the implants during coating, and coating permeability adjusters.

The present compositions may additionally contain one or more optional additives such as fibrous reinforcement, colorants, perfumes, rubber modifiers, modifying agents, etc. In practice, each of these optional additives should be compatible with the resulting polymer and its intended use. Examples of suitable fibrous reinforcement include PGA microfibrils, collagen microfibrils, cellulosic microfibrils, and olefinic microfibrils. The amount of each of these optional additives employed in the composition is an amount necessary to achieve the desired effect.

Physical Structures of the Subject Compositions

The subject polymers may be formed in a variety of shapes. For example, in certain embodiments, subject polymer matrices may be presented in the form of microparticles or nanoparticles. Such particles may be prepared by a variety of methods known in the art, including for example, solvent evaporation, spray-drying or double emulsion methods.

The shape of microparticles and nanoparticles may be determined by scanning electron microscopy. Spherically shaped nanoparticles are used in certain embodiments for circulation through the bloodstream. If desired, the particles may be fabricated using known techniques into other shapes that are more useful for a specific application.

In addition to intracellular delivery of an antineoplastic taxane, it also possible that particles of the subject compositions, such as microparticles or nanoparticles, may undergo endocytosis, thereby obtaining access to the cell. The frequency of such an endocytosis process will likely depend on the size of any particle.

In certain embodiments, solid articles useful in defining shape and providing rigidity and structural strength to the polymeric matrices may be used. For example, a polymer may be formed on a mesh or other weave for implantation. A polymer may also be fabricated as a stent or as a shunt, adapted for holding open areas within body tissues or for draining fluid from one body cavity or body lumen into another. Further, a polymer may be fabricated as a drain or a tube suitable for removing fluid from a post-operative site, and in some embodiments adaptable for use with closed section drainage systems such as Jackson-Pratt drains and the like familiar in the art. In prostate cancer patients, fabrications consistent with the present invention may be employed as stents to enhance urethral patency in those cases where strictures related to the prostate cancer or its treatments are anticipated.

The mechanical properties of the polymer may be important for the processability of making molded or pressed articles for implantation. For example, the glass transition temperature may vary widely but must be sufficiently lower than the temperature of decomposition to accommodate conventional fabrication techniques, such as compression molding, extrusion or injection molding.

Biodegradability and Release Characteristics

In certain embodiments, the polymers and blends of the present invention, upon contact with body fluids, undergo gradual degradation. The life of a biodegradable polymer in vivo depends, among other things, upon its molecular weight, crystallinity, biostability, and the degree of crosslinking. In general, the greater the molecular weight, the higher the degree of crystallinity, and the greater the biostability, the slower biodegradation will be.

If a subject polymer matrix is formulated with an antineoplastic agent, release of such an agent for a sustained or extended period as compared to the release from an isotonic saline solution generally results. Such release profile may result in prolonged delivery (over, say 1 to about 4,000 hours, or alternatively about 4 to about 1500 hours) of effective amounts (e.g., about 0.00001 mg/kg/hour to about 10 mg/kg/hour) of the agent associated with the polymer.

A variety of factors may affect the desired rate of hydrolysis of polymers of the subject invention, the desired softness and flexibility of the resulting solid matrix, rate and extent of bioactive material release. Some of such factors include: the selection of the various substituent groups, such as the phosphate group making up the linkage in the polymer backbone (or analogs thereof), the enantiomeric or diastereomeric purity of the monomeric subunits, homogeneity of subunits found in the polymer, and the length of the polymer. For instance, the present invention contemplates heteropolymers with varying linkages, and/or the inclusion of other monomeric elements in the polymer, in order to control, for example, the rate of biodegradation of the matrix.

To illustrate further, a wide range of degradation rates may be obtained by adjusting the hydrophobicities of the backbones or side chains of the polymers while still maintaining sufficient biodegradability for the use intended for any such polymer. Such a result may be achieved by varying the various functional groups of the polymer. For example, the combination of a hydrophobic backbone and a hydrophilic linkage produces heterogeneous degradation because cleavage is encouraged whereas water penetration is resisted. In another example, it is expected that use of substituent on phosphate in the polymers of the present invention that is lipophilic, hydrophobic or bulky group would slow the rate of degradation. For example, it is expected that conversion of the phosphate side chain to a more lipophilic, more hydrophobic or more sterically bulky group would slow down the rate of biodegradation. Thus, release is usually faster from polymer compositions with a small aliphatic group side chain than with a bulky aromatic side chain.

One protocol generally accepted in the field that may be used to determine the release rate of any antineoplastic agent or other material loaded in the polymer matrices of the present invention involves degradation of any such matrix in a 0.1 M PBS solution (pH 7.4) at 37° C., an assay known in the art. For purposes of the present invention, the term “PBS protocol” is used herein to refer to such protocol.

In certain instances, the release rates of different polymer systems of the present invention may be compared by subjecting them to such a protocol. In certain instances, it may be necessary to process polymeric systems in the same fashion to allow direct and relatively accurate comparisons of different systems to be made. For example, the present invention teaches several different means of formulating the polymeric matrices of the present invention. Such comparisons may indicate that any one polymeric system releases incorporated material at a rate from about 2 or less to about 1000 or more times faster than another polymeric system. Alternatively, a comparison may reveal a rate difference of about 3, 5, 7, 10, 25, 50, 100, 250, 500 or 750. Even higher rate differences are contemplated by the present invention and release rate protocols.

In certain embodiments, when formulated in a certain manner, the release rate for polymer systems of the present invention may present as mono- or bi-phasic. Release of any material incorporated into the polymer matrix, which is often provided as a microsphere, may be characterized in certain instances by an initial increased release rate, which may release from about 5 to about 50% or more of any incorporated material, or alternatively about 10, 15, 20, 25, 30 or 40%, followed by a release rate of lesser magnitude.

The release rate of any incorporated material may also be characterized by the amount of such material released per day per mg of polymer matrix. For example, in certain embodiments, the release rate may vary from about 1 ng or less of any incorporated material per day per mg of polymeric system to about 5000 or more ng/day/mg. Alternatively, the release rate may be about 10, 25, 50, 75, 100, 125, 150, 175, 200, 250, 300, 350, 400, 450, 500, 600, 700, 800 or 900 ng/day/mg. In still other embodiments, the release rate of any incorporated material may be 10,000 ng/day/mg or even higher. In certain instances, materials incorporated and characterized by such release rate protocols may include antineoplastic agents, fillers, and other substances.

In another aspect, the rate of release of any material from any polymer matrix of the present invention may be presented as the half-life of such material in the such matrix.

In addition to the embodiment involving protocols for in vitro determination of release rates, in vivo protocols, whereby in certain instances release rates for polymeric systems may be determined in vivo, are also contemplated by the present invention. Other assays useful for determining the release of any material from the polymers of the present system are known in the art.

4. Implants and Delivery Systems

In its simplest form, a delivery system for an antineoplastic agent for treatment of prostate cancer consists of a dispersion of such an agent into one of the polymers described above. In other embodiments, an article is used for implantation, injection, or otherwise placed totally or partially within the body, the article comprising a composition for treatment of prostate cancer. It may be particularly important that such an article result in minimal tissue irritation when applied to, implanted in or injected into vascularized tissue, hypovascularized post-operative tissue or tissue exposed to previous radiation that is part of the prostate. In certain embodiments, a solid, flowable or fluid article comprising the composition of the invention is inserted within an anatomic area by implantation, injection, endoscopy or otherwise being placed within an anatomic area of the subject being treated for a prostate cancer.

As a structural medical device, the polymer compositions of the inventions provide a wide variety of physical forms having specific chemical, physical and mechanical properties suitable for insertion into an anatomic area.

Biocompatible delivery systems and articles thereof, may be prepared in a variety of ways known in the art. The subject polymer may be melt processed using conventional extrusion or injection molding techniques, or these products may be prepared by dissolving in an appropriate solvent, followed by formation of the device, and subsequent removal of the solvent by evaporation or extraction, e.g., by spray drying. By these methods, the polymers may be formed into articles of almost any size or shape desired, for example, implantable solid discs or wafers or injectable rods, microspheres, or other microparticles. Typical medical articles also include such as implants as laminates for degradable fabric or coatings to be placed on other implant devices.

In one embodiment, certain polymer compositions of the subject invention may be used to form a soft, drug-delivery “depot” that can be administered as a liquid, for example, by injection, but which remains sufficiently viscous to maintain the drug within the localized area around the injection site. By using a polymer composition in flowable form, even the need to make an incision can be eliminated. In any event, the flexible or flowable delivery “depot” will adjust to the shape of the space it occupies within the body with a minimum of trauma to surrounding tissues.

When the polymer composition of the invention is flexible or flowable, it may be placed anywhere within the body, including into an anatomic area of the prostate. It may be inserted into the anatomic area either through an open surgical wound, under direct or indirect vision, or through any of the access devices routinely used in the art to enter such areas, for example, indwelling or acutely-inserted catheters, needles, drains, superselective angiography means and the like. A flowable or fluid polymer may be adapted for mixing with the transudate or exudate found within or expected to gather within the anatomic area. A flowable or fluid polymer may be instilled in an anatomic area during surgery on organs or structures therein to decrease the likelihood of recurrent disease when there is a high risk for its development. In certain embodiments, a polymer composition according to the present invention may also be incorporated in access devices so that an antineoplastic agent is released into the anatomic area within which the access device resides, thereby decreasing the size of a primary or recurrent prostate cancer, treating said cancer, or preventing the development of recurrent disease where a cancer has been extirpated. The polymer composition of the invention may also be used to produce coatings for other solid implantable devices for treatment of prostate cancer.

Once a system or implant article is in place, it should remain in at least partial contact with a biological fluid, such as blood, tissue fluid, lymph, urine, or secretions from organ surfaces or mucous membranes, and the like to allow for sustained release of any encapsulated therapeutic agent, e.g., an antineoplastic agent.

5. Exemplary Methods of Making the Subject Polymers

In general, the polymers of the present invention may be prepared by melt polycondensation, solution polymerization or interfacial polycondensation. Techniques necessary to prepare the subject polymers are known in the art, and reference is made in particular to U.S. patent application Ser. No. 09/885,085, filed Jun. 21, 2001, which is hereby incorporated by this reference in its entirety.

The most common general reaction in preparing the subject compositions is a dehydrochlorination between a phosphodichloridate and a diol according to the following equation:

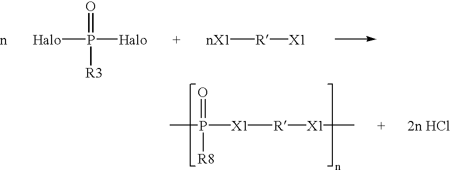

Certain of the subject polymers may be obtained by condensation between appropriately substituted dichlorides and diols.

An advantage of melt polycondensation is that it avoids the use of solvents and large amounts of other additives, thus making purification more straightforward. This method may also provide polymers of reasonably high molecular weight. Somewhat rigorous conditions, however, are often required and may lead to chain acidolysis (or hydrolysis if water is present). Unwanted, thermally induced side reactions, such as cross-linking reactions, may also occur if the polymer backbone is susceptible to hydrogen atom abstraction or oxidation with subsequent macroradical recombination.

To minimize these side reactions, the polymerization may also be carried out in solution. Solution polycondensation requires that both the prepolymer and the phosphorus component be sufficiently soluble in a common solvent. Typically, a chlorinated organic solvent is used, such as chloroform, dichloromethane or dichloroethane. The solution polymerization is generally run in the presence of equimolar amounts of the reactants and, in one embodiment, an excess of an acid acceptor and a catalyst, such as 4-dimethylaminopyridine (DMAP). Useful acid acceptors include tertiary amines as pyridine or triethylamine. The product is then typically isolated from the solution by precipitation in a non-solvent and purified to remove the hydrochloride salt by conventional techniques known to those of ordinary skill in the art, such as by washing with an aqueous acidic solution, e.g., dilute HCl.

Reaction times tend to be longer with solution polymerization than with melt polymerization. However, because overall milder reaction conditions may be used, side reactions are minimized, and more sensitive functional groups may be incorporated into the polymer. The disadvantages of solution polymerization are that removal of solvents may be difficult.

Interfacial polycondensation may be used when high molecular-weight polymers are desired at high reaction rates. By such methods, mild conditions minimize side reactions, and the dependence of high molecular weight on stoichiometric equivalence between diol and dichloridate inherent in solution methods is removed. However, hydrolysis of the acid chloride may occur in the alkaline aqueous phase, and sensitive dichloridates that have some solubility in water are generally subject to hydrolysis rather than polymerization. Phase transfer catalysts, such as crown ethers or tertiary ammonium chloride, may be used to bring the ionized diol to the interface to facilitate the polycondensation reaction. The yield and molecular weight of the resulting polymer after interfacial polycondensation are affected by reaction time, molar ratio of the monomers, volume ratio of the immiscible solvents, the type of acid acceptor, and the type and concentration of the chase transfer catalyst.

Methods for making the present invention may take place at widely varying temperatures, depending upon whether a solvent is used and, if so, which one; the molecular weight desired; the susceptibility of the reactants to form side reactions; and the presence of a catalyst. Usually, the process takes place at a temperature ranging from about 0 to about +235° C. for melt conditions. Somewhat lower temperatures, e.g., for example from about −50 to about 100° C., may be possible with solution polymerization or interfacial polycondensation with the use of either a cationic or anionic catalyst.

The time required for the process may vary widely, depending on the type of reaction being used, the molecular weight desired and, in general, the need to use more or less rigorous conditions for the reaction to proceed to the desired degree of completion. Typically, however, the synthetic process takes place during a time between about 30 minutes and about 7 days.

Although the process may be in bulk, in solution, by interfacial polycondensation, or any other convenient method of polymerization, in many instant embodiments, the process takes place under solution conditions. Particularly useful solvents include methylene chloride, chloroform, tetrahydrofuran, di-methyl formamide, dimethyl sulfoxide or any of a wide variety of inert organic solvents.

In greater detail, polymers of Formula VI may be prepared, at least in part, by reacting a compound having a formula H-Y1-L1-Y1-H, such as 2-aminoethanol, ethylene glycol, ethane dithiol, etc., with a cyclic compound, e.g., having one of the following structures: for example, caprolactone or lactide (lactic acid dimer).

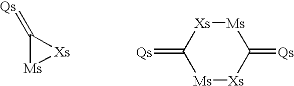

Thus, the cyclic compound may include one or two subunits ts. For cyclic compounds containing two subunits, the two subunits contained therein may be the same or different.

For synthesizing, for example, a compound of Formula VI, wherein x and y are on average about 10, an equivalent of ethylene glycol as H-Y1-L1-Y1-H may be reacted with 20 equivalents of

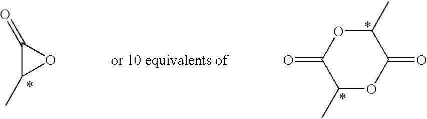

because lactic acid dimer contains two monomer units for each equivalent of the cyclic compound. Variation of the ratio of cyclic compound to ethylene glycol or other bifunctional core will likewise vary the values of x and y, although x and y will be substantially equal for a symmetrical bifunctional core (e.g., ethylene glycol) for subject polymers prepared by this method. For an unsymmetrical bifunctional core (e.g., 2-aminoethanol), the ratio of x:y may vary considerably, as will be understood by one of skill in the art and may be determined without undue experimentation.

Polymers of the present invention may generally be isolated from the reaction mixture by conventional techniques, such as by precipitating out, extraction with an immiscible solvent, evaporation, filtration, crystallization and the like. Typically, the subject polymers are both isolated and purified by quenching a solution of polymer with a non-solvent or a partial solvent, such as diethyl ether or petroleum ether.

In certain embodiments, the subject polymers are soluble in one or more common organic solvents for ease of fabrication and processing. Common organic solvents include such solvents as chloroform, dichloromethane, dichloroethane, 2-butanone, butyl acetate, ethyl butyrate, acetone, ethyl acetate, dimethylacetamide, N-methyl pyrrolidone, dimethylformamide, and dimethylsulfoxide.

6. Exemplary Methods for Treating Prostate Cancers

As contemplated by the present invention, the antineoplastic agent for treatment of prostate cancer will be released from a subject polymer system in an amount sufficient to deliver to a patient a therapeutically effective amount of such agent as part of a prophylactic or therapeutic treatment. The desired concentration of active compound in the polymer system will depend on absorption, inactivation, and excretion rates of the drug as well as the delivery rate of the compound from the subject composition. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art. As one non-limiting example, dosage may be based on the amount of the antineoplastic agent encapsulated in the subject polymers. For example, a range of amounts of antineoplastic agent are contemplated, including 0.5, 1, 2, 3, 4, 5, 7.5, 10, 15, 20, 25 mg or more of such agent per kg body weight of the patient. Other amounts will be known to those of skill in the art and readily determined.

Methods for treating prostate cancers according to the present invention involve gaining access to an anatomic area where a prostate cancer to be treated is located or may grow and instilling therein a composition comprising a biocompatible, and optionally biodegradable polymer and an antineoplastic agent. In certain embodiments, the antineoplastic agent is an antineoplastic taxane. According to the present invention, in certain embodiments the polymer composition may be a fluid, a flowable material or a rigid or flexible solid article. Access to the anatomic area is gained by techniques familiar to practitioners in the medical arts. In certain embodiments, the compositions of the present invention are instilled into the anatomic area to prevent or to minimize the occurrence or recurrence of a prostate cancer in a patient who is at increased risk for developing such a disease. Optionally, the polymeric composition of the present composition may be removed at a preselected time interval after it is instilled, although certain compositions according to the present invention are formulated to reside within the anatomic for prolonged periods of time or permanently, in certain cases degrading over time or being resorbed by, digested by or metabolized by the local body tissues. Repeated instillations of the subject polymeric compositions may be undertaken, but certain compositions are formulated for sustained or extended release of the therapeutically effective amount of antineoplastic agent, so that a single applied dose may be sufficient to treat the malignant effusion adequately. Combination therapies for advanced prostate cancer patients also fall within the scope of the present invention where one component of the combination therapy involves the instillation of the compositions of the present invention as claimed and as described herein. As an example, combined treatment regimens may involve the instillation of an antineoplastic agent within an anatomic area accompanied by another type of treatment, such as systemic chemotherapy administration or locoregional radiation therapy, cryotherapy or other therapeutic application of electromagnetic energy. Other therapeutic combinations, all falling similarly within the scope of the present invention, will be apparent to practitioners of ordinary skill in the art using no more than routine experimentation. For example, and without limitation, the modalities of the therapeutic combination may have an affect on result of treatment, such as the timing of radiation treatment.

Certain exemplary treatment methods for various aspects of prostate cancer are described below. It is understood, however, that these descriptions are intended as illustrative only, not intended to be limiting in any way, and that other modifications and variations of these illustrative embodiments may be contemplated without departing from the scope of the present invention.

Instillation of compositions according to these inventive methods may accompany procedures for resecting prostate cancers or other surgical procedures. Furthermore, these methods are consistent with prophylactic application, in those cases where the risk of developing primary or recurrent prostate cancer is high. For example, in a surgical procedure where extensive disease is apparent, the clinician might deem it advisable to apply the compositions of the present invention around the excisional area using any of the delivery systems that would be familiar in the art. A liquid, gel, spray, aerosol or formed article could be used under these circumstances to deploy the inventive compositions for the prevention or the minimization of recurrent disease in the future. The compositions of the present invention may be suitable for implantation into sites where retropubic or perineal prostatectomy has been performed, or may be suitable for implantation into prostatic tissue affected with benign hyperplasia where cellular predisposition to developing malignancy may be identified. A delivery system adapted to any of these treatments for prostate cancer could be fabricated and composed to carry out other desirable medical functions without exceeding the scope of the present invention: for example, an antineoplastic taxane composition according to the present invention could be combined with other substances such as anti-adhesion substances, hemostatic substances, immunogenic substances, or any other therapeutic agent without limitation. Materials bearing the inventive compositions may also be adapted for activation using electromagnetic radiation, including heat energy, light energy and therapeutic radiation delivered from internal or external sources.

The efficacy of treatment with the subject compositions may be determined in a number of fashions. In one method, the median survival rate or median survival time or life span for treatment with a subject composition may be compared to other forms of treatment with the same antineoplastic agent. The increase in median survival rate or time or life span for treatment with a subject composition as compared to treatment with another method may be 10, 25, 50, 75, 100, 150, 200, 300, 400% or even more. The period of time for observing any such increase may be about fifteen days, three months, six months, one year, three years, or five or more years. The comparison may be made against treatment with the antineoplastic agent itself, or administration of the agent in a pharmaceutically acceptable carrier, or administration as part of a different drug delivery device than a subject composition. The comparison may be made against the same or a different effective dosage of the antineoplastic agent. The different regiments compared may use electromagnetic radiation.

Alternatively, the different treatment regimens described above may be compared by comparing tumor volume doubling times, with the length of time required for tumor volume to double being approximately two-thirds, one-half, one-third, one-quarter, one-fifth, one-tenth, one-twentieth or even less for treatment with a subject composition as compared to treatment with another method using the same antineoplastic agent.

Alternatively, a comparison of the different treatment regimens described above may be based on the effectiveness of the treatment, with treatment with a subject composition being substantially better, or 50%, 100%, 150%, 200%, 300% more effective, than by another method using the same antineoplastic agent.

Alternatively, the different treatment regimens may be analyzed by comparing the therapeutic index for each of them, with treatment with a subject composition as compared to another regimen having a therapeutic index two, three, five or seven times that of, or even one, two, three or more orders of magnitude greater than, treatment with another method using the same antineoplastic agent.

Alternatively, the different treatment regimens may be analyzed by comparing the frequency of hypersensitivity reactions to each of them, with treatment with a subject composition reducing the number of hypersensitivity reactions by at least about 10, 25, 50, 75, 100, 150, 200 or even more percent as compared to another method using the same antineoplastic agent. Such comparisons may take into account whether the hypersensitivity reaction is significant and whether premedication is used

7. References

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

Patents

U.S. Pat. Nos. 4,638,045, 5,219,564, 5,099,060, 6,040,330, 6,017,935, 6,002,023, 5,990,325, 5,981,564, 5,977,164, 5,977,163, 5,972,992, 5,922,754, 5,919,815, 5,908,835, 5,912,263, 5,902,822, 5,877,205, 5,854,278, 5,840,929, 5,821,363, 5,817,840, 5,808,888, 5,795,909, 5,780,653, 5,773,464, 5,773,461, 5,767,297, 5,767,296, 5,760,072, 5,756,776, 5,750,691, 5,739,359, 5,728,687, 5,719,177, 5,693,666, 5,688,977, 5,686,623, 5,670,536, 5,614,645, 5,608,087, 5,597,931, 5,908,835, 6,005,120, 5,424,073, and 5,547,981.

### PUBLICATIONS AND OTHER REFERENCES

Ertel et al., (1995) J. Biomedical Materials Res. 29:1337-1348

Choueka et al., (1996) J. Biomed. Materials Res., 31:35-41)

Langer et al., (1983) Rev. Macro. Chem. Phys. C23(1):61

Leong et al. (1986) Biomaterials, 7:364

Sato et al., (1996) Bio. Pharm. Bull. 19:1596-601

8. Exemplification

The invention now being generally described, it will be more readily understood by reference to the following examples which are included merely for purposes of illustration of certain aspects and embodiments of the present invention and are not intended to limit the invention in any way.

### EXAMPLE 1

### First Synthesis of D,L-PL(PG)EOP

All glassware was dried for a minimum of 2 hours at 105° C. and allowed to cool in a desiccator or cooled under a stream of argon gas. A 28.5 g portion of D,L-lactide and 1.5 g of 1,2-propanediol (PG), obtained from Aldrich, Catalog No. 39,803, 99.5+%, in a molar ratio of 10:1, were weighed into a 250 mL 3-neck round-bottom flask. The flask was equipped with a gas joint and a stirrer bearing/shaft/paddle assembly. The mixture was evacuated and pressurized with argon five times to remove residual air and moisture. The reaction apparatus was immersed in a preheated oil bath at 135° C., connected to an argon source with an oil bubbler, and stirred at a moderate speed until all of the solid monomer had melted.

At this time, a volume of stock stannous octoate solution (about 130 mg/ml in toluene of chloroform) equivalent to 3.6 mg tin (120 ppm stannous octoate or equivalent to 35 ppm tin based upon weight of the prepolymer) was added to the melt using a 50 μL syringe. The reaction mixture was allowed to stir under a slight argon pressure for approximately 16 hours. The oil bath temperature was then reduced to about 110° C. and the residual monomer was removed under vacuum. The upper parts of the reaction assembly were heated gently with a heat gun to aid in the monomer removal. The total time under vacuum was 2-3 hours. A reflux condenser was then inserted between the gas joint and the flask in the prepolymer apparatus described above. The molten prepolymer was dissolved by adding 100 mL of chloroform to the reaction flask with stirring.

Next, 6.9 mL of triethylamine (TEA) and 1.21 g of DMAP were added to the stirring reaction mixture. The reaction mixture was then chilled to about 4° C. in an ice bath. A solution of approximately 2.5 mL of freshly distilled ethyl dichlorophosphate (EOPCl₂) in 25 mL of chloroform was prepared in a dropping funnel. The solution in the funnel was added drop wise to the reaction mixture over a period of about 30 minutes. After the addition was complete the reaction mixture was allowed to continue stirring at about 4° C. for 10 minutes and then the ice bath was removed. The reaction mixture was allowed to warm to room temperature over about 1 hour. At this time a significant increase in viscosity of the clear solution was observed. The reaction mixture was then heated to reflux using an oil bath. Over the next hour the solution became cloudy. The reaction mixture was allowed to reflux over two nights, about 38 hours total.

At this time, a Barret trap was inserted between the condenser and the flask and 88 mL of solvent (⅔ of the total volume) were distilled from the reaction mixture. The Barret trap was removed and the reaction mixture was allowed to reflux for an additional 16 hours with the oil bath temperature between 98-102° C. Next, the oil bath temperature was increased to 115° C. for 2 hours. After this time, the reaction mixture was allowed to cool to room temperature, and 200 mL of dichloromethane was added and transferred to a separatory funnel. The reaction mixture was extracted twice with 100 mL of 0.1 M HCl and twice with 100 mL of saturated sodium chloride solution. The organic layer was isolated, dried overnight in the freezer at about −15° C. over 50 g of sodium sulfate, and filtered twice. The resulting polymer solution was poured into 1500 mL of hexane plus 500 mL of ether. The resulting mass of polymer was dried under vacuum. The Inherent Viscosity (IV) of this material was measured to be 0.39 dL/g.

### EXAMPLE 2

### Second Synthesis of D,L-PL(PG)EOP

All glassware was dried for a minimum of 2 hours at 105° C. and allowed to cool in a desiccator or cooled under a stream of argon gas. A 28.5 g portion of D,L-lactide and 1.5 g of PG (molar ratio, 10:1) were weighed into a 250 ml 3-neck round-bottom flask. The flask was equipped with a gas joint and a stirrer bearing/shaft/paddle assembly. The mixture was evacuated and filled with argon five times to remove residual air and moisture. Each time the polymerization vessel was evacuated to a pressure between 0.5 and 10 Torr. The reaction apparatus was immersed in a preheated oil bath at 125° C., connected to an argon source with an oil bubbler, and stirred at a moderate speed until all of the solid monomer had melted. At this time, a volume of stock stannous octoate solution (about 130 mg/ml in toluene) equivalent to 100 ppm stannous octoate (29 ppm Sn) was added to the melt using a syringe. The reaction mixture was allowed to stir under a slight argon pressure for 3 hours. The oil bath temperature was then reduced to about 105° C. and the residual monomer was removed under vacuum. The pressure was maintained as low as possible, typically between 0.5 and 10 Torr. The upper parts of the reaction assembly were heated gently with a heat gun to aid in the monomer removal. The total time under vacuum was 1 hour.

The prepolymer was cooled to room temperature under argon gas and allowed to stand for 12-18 hours at ambient temperature. The prepolymer was dissolved in 84 ml of chloroform with stirring and 2.5 equivalents of triethylamine (TEA) and 0.5 equivalents of DMAP were added to the stirring reaction mixture using a powder funnel. The reaction mixture was chilled to about −5 to about −15° C. in a cold bath. A solution of about 1 equivalent of distilled ethyl dichlorophosphate (EOPCl₂) in 10 ml of chloroform was prepared in a dropping funnel. The solution in the funnel was added slowly to the reaction mixture over a period of 0.5 hour.

After the addition was complete, the reaction mixture was allowed to stir at low temperature for 1 hour at −5° C. The reaction was then quenched with 1 ml of anhydrous methanol and stirred for another five minutes. Next, the reaction mixture was transferred to a 0.5 gallon vessel and mixed with 37 g of Dowex DR-2030 IER and 30 g of Dowex M-43, and shaken on a mechanical shaker for 2 hour to remove residual DMAP and TEA free base and salts (the IERs had been washed with several bed volumes of methanol and chloroform and dried under vacuum at ambient temperature for about 18 hours). The resin was removed from the reaction mixture by vacuum filtration through Whatman 54 filter paper.

The resin was washed with about one bed volume of dichloromethane and the filtrate was concentrated to approximately 50 ml. The viscous filtrate was poured into 200 ml of petroleum ether to precipitate the polymer. The polymer mass was washed with 100 ml of petroleum ether and dried under vacuum. Molecular weights of the polymers were obtained from gel permeation chromatography (GPC) using both differential refractive index detection and a polystyrene calibration curve (CC) and by light scattering detection. The molecular weight and IV data for the polymers prepared by this process are listed in the table below.

| Sample | Mw (LS), daltons | Mw (CC), daltons | IV, dL/g |
|---|---|---|---|
| 1 | 101,200 | 107,500 | 0.62 |
| 2 | 150,100 | 155,900 | 0.80 |
| 3 | 85,200 | 84,300 | — |
| 4 | 92,600 | 89,900 | — |

### EXAMPLE 3

### Synthesis of D,L-PL(EG)FOP

All glassware was dried for a minimum of 2 hours at 105° C. and allowed to cool in a desiccator or cooled under a stream of argon gas. A 100.0 g portion of D,L-lactide and 4.3 g of ethylene glycol (EG) (molar ratio, 10:1) were weighed into a 1000 ml 3-neck round-bottom flask. The flask was equipped with a gas joint and a stirrer bearing/shaft/paddle assembly. The mixture was evacuated and filled with argon five times to remove residual air and moisture. The reaction apparatus was immersed in a preheated oil bath at 135° C., connected to an argon source with an oil bubbler, and stirred at a moderate speed until all of the solid monomer had melted.

At this time, a volume of stock stannous octoate solution (about 130 mg/ml in toluene) equivalent to 120 ppm stannous octoate or 35 ppm Sn was added to the melt using a syringe. The reaction mixture was allowed to stir under a slight argon pressure for approximately 16 hours. The oil bath temperature was then reduced to about 110° C. and the residual monomer was removed under vacuum. The upper parts of the reaction assembly were heated gently with a heat gun to aid in the monomer removal. The total time under vacuum was 2-3 hours.

The molten prepolymer was dissolved in 350 ml of chloroform with stirring and 2.5 equivalents of TEA and 0.5 equivalents of DMAP were added to the stirring reaction mixture using a powder funnel. The reaction mixture was chilled to about −5° C. in a cold bath. A solution of about 1 equivalent of distilled ethyl dichlorophosphate (EOPCl₂) in 97 ml of chloroform was prepared in a dropping funnel. The solution in the funnel was added slowly to the reaction mixture over a period of 2 hours. After the addition was complete, the reaction mixture was allowed to stir at low temperature for 45 minutes at -5° C. After 2 hours a significant increase in viscosity of the clear solution was observed. The reaction was then quenched with 6.8 ml of anhydrous methanol and stirred for another five minutes.

Next, the reaction mixture was transferred to a 0.5 gallon vessel and mixed with 87 g of Dowex HCR-S IER and 104 g of Dowex-43, and shaken on a mechanical shaker for 1 hour to remove residual DMAP and TEA free base and salts (the IERs had been washed with several bed volumes of methanol and dried under vacuum at ambient temperature for about 18 hours). The resin was removed from the reaction mixture by vacuum filtration through Whatman 54 filter paper. The resin was washed with about one bed volume of dichloromethane and the filtrate was concentrated to approximately 150 ml. The viscous filtrate was poured into 2000 ml of hexane to precipitate the polymer. The polymer mass was washed with 2×200 ml of hexane and dried under vacuum. The molecular weights were determined by GPC were 40,400 for Mw (LS) and 42,000 for Mw (CC).

### EXAMPLE 4

### Synthesis of D,L-PL(HD)EOP

All glassware was dried for a minimum of 2 hours at 105° C. and allowed to cool in a desiccator or cooled under a stream of argon gas. A 100.0 g portion of D,L-lactide and 8.2 g of 1,6-hexane diol (HD) (molar ratio, 10:1) were weighed into a 1000 ml 3-neck round-bottom flask. The flask was equipped with a gas joint and a stirrer bearing/shaft/paddle assembly. The mixture was evacuated and filled with argon five times to remove residual air and moisture. The reaction apparatus was immersed in a preheated oil bath at 135° C., connected to an argon source with an oil bubbler, and stirred at a moderate speed until all of the solid monomer had melted.

At this time, a volume of stock stannous octoate solution equivalent (about 130 mg/ml in toluene) to 120 ppm stannous octoate or 35 ppm Sn was added to the melt using a syringe. The reaction mixture was allowed to stir under a slight argon pressure for approximately 16 hours. The oil bath temperature was then reduced to about 110° C. and the residual monomer was removed under vacuum. The upper parts of the reaction assembly were heated gently with a heat gun to aid in the monomer removal. The total time under vacuum was 2-3 hours.

The molten prepolymer was dissolved in 350 ml of chloroform with stirring and 2.5 equivalents of triethylamine (TEA) and 0.5 equivalents of DMAP were added to the stirring reaction mixture using a powder funnel. The reaction mixture was chilled to about −5° C. in a cold bath. A solution of about 1 equivalent of distilled ethyl dichlorophosphate (EOPCl₂) in 97 ml of chloroform was prepared in a dropping funnel. The solution in the funnel was added slowly to the reaction mixture over a period of 2 hours. After the addition was complete, the reaction mixture was allowed to stir at low temperature for 45 minutes at −5° C. After 2 hours, a significant increase in viscosity of the clear solution was observed. The reaction was then quenched with 6.8 ml of anhydrous methanol and stirred for another five minutes.

Next, the reaction mixture was transferred to a 0.5 gallon vessel and mixed with 87 g of Dowex HCR-S IER and 104 g of Dowex-43, and shaken on a mechanical shaker for 1 hour to remove residual DMAP and TEA free base and salts (the IERs had been washed with several bed volumes of methanol and dried under vacuum at ambient temperature for about 18 hours). The resin was removed from the reaction mixture by vacuum filtration through Whatman 54 filter paper. The resin was washed with about one bed volume of dichloromethane and the filtrate was concentrated to approximately 150 ml. The viscous filtrate was poured into 2000 ml of hexane to precipitate the polymer. The polymer mass was washed with 2×200 ml of hexane and dried under vacuum. The molecular weights were determined by GPC were 36,700 for Mw (LS) and 34,100 for Mw (CC). The value for IV was 0.33 dL/g.

### EXAMPLE 5

### Polymer of PG, D,L-lactide, Glycolide and Ethyl Dichlorophosphate

All glassware was dried for a minimum of 2 hours at 105° C. and allowed to cool in a desiccator or cooled under a stream of argon gas. A 28.5 g portion of D,L-lactide and 1.5 g of PG (molar ratio, 10:1) were weighed into a 250 ml 3-neck round-bottom flask. The flask was equipped with a gas joint and a stirrer bearing/shaft/paddle assembly and a 125 ml dropping funnel containing 4.6 g of glycolide. The mixture was evacuated and filled with argon five times to remove residual air and moisture. The reaction apparatus was immersed in a preheated oil bath at 135° C., connected to an argon source with an oil bubbler, and stirred at a moderate speed until all of the solid monomer had melted.

At this time, a volume of stock stannous octoate solution (about 130 mg/ml in toluene) equivalent to 3.6 mg tin (120 ppm stannous octoate or 35 ppm tin) was added to the melt using a 50 μl syringe. The reaction mixture was allowed to stir under a slight argon pressure for approximately 16 hours. At this time the glycolide was melted using a heat gun and added to the polymer melt in the flask. The melt was stirred for an additional 2 hours. The oil bath temperature was then reduced to about 115° C. and the residual monomer was removed under vacuum. The upper parts of the reaction assembly were heated gently with a heat gun to aid in the monomer removal. The total time under vacuum was 2 hours.

The molten prepolymer was suspended in 84 ml of chloroform with stirring and 2. 5 equivalents of TEA and 0.5 equivalents of DMAP were added to the stirring reaction mixture using a powder funnel. The reaction mixture was chilled to about 4° C. in a cold bath. A solution of about 1 equivalent of distilled ethyl dichlorophosphate (EOPCl₂) in 27.5 ml of chloroform was prepared in a dropping funnel. The solution in the funnel was added slowly to the reaction mixture over a period of 1 hour. After the addition was complete, the reaction mixture was allowed to stir at low temperature for another 1.75 hours and then the cold bath was removed. The reaction mixture was allowed to warm to room temperature and stirred for 2 to 18 hours. After 2 hours a significant increase in viscosity of the clear solution was observed. The reaction was then quenched with 1 ml of anhydrous methanol and stirred for another five minutes.

Next, 37 g of dry Dowex HCR-S IER and 30 g of dry Dowex M-43 were added to the reaction mixture and stirring was continued for another hour to remove residual DMAP and TEA free base and salts. The IERs were removed from the reaction mixture by vacuum filtration through Whatman 54 filter paper. The resin was washed with about one bed volume of dichloromethane and the filtrate was concentrated to approximately 50 ml. The viscous filtrate was poured into 700 ml of petroleum ether to precipitate the polymer and dried under vacuum.

### EXAMPLE 6

### Synthesis of D,L-PL(PG)HOP

All glassware was dried for a minimum of 2 hours at 105° C. and allowed to cool in a desiccator or cooled under a stream of argon gas. A 28.5 g portion of D,L-lactide and 1.5 g of PG (molar ratio, 10:1) were weighed into a 250 ml 3-neck round-bottom flask. The flask was equipped with a gas joint and a stirrer bearing/shaft/paddle assembly. The mixture was evacuated and filled with argon five times to remove residual air and moisture. The reaction apparatus was immersed in a preheated oil bath at 135° C., connected to an argon source with an oil bubbler, and stirred at a moderate speed until all of the solid monomer had melted.

At this time, a volume of stock stannous octoate solution (about 130 mg/ml in toluene) equivalent to 3.6 mg tin (120 ppm stannous octoate or 35 ppm tin) was added to the melt using a 50 μl syringe. The reaction mixture was allowed to stir under a slight argon pressure for approximately 16 hours. The oil bath temperature was then reduced to about 110° C. and the residual monomer was removed under vacuum. The upper parts of the reaction assembly were heated gently with a heat gun to aid in the monomer removal. The total time under vacuum was 2-3 hours.

The molten prepolymer was dissolved in 100 ml of chloroform with stirring and TEA and DMAP were added to the stirring reaction mixture using a powder funnel. The funnel was rinsed with 10 ml of chloroform. The reaction mixture was chilled to about 4° C. in a cold bath. A solution of about 1 equivalent of distilled hexyl dichlorophosphate (HOPCl₂) in 27.5 ml of chloroform was prepared in a dropping funnel. The solution in the funnel was added slowly to the reaction mixture over a period of 1 hour. After the addition was complete, the reaction mixture was allowed to stir at low temperature for another hour and then the cold bath was removed. The reaction mixture was allowed to warm to room temperature and stirred for 2 to 18 hours. After 2 hours a significant increase in viscosity of the clear solution was observed. The reaction was then quenched with 800 μl of anhydrous methanol and stirred for another five minutes.

Next, Dowex MR-3C ion exchange resin (IER) was added to the reaction mixture and stirring was continued for another hour to remove residual DMAP and TEA free base and salts (the Dowex resin had been washed with several bed volumes of methanol and dried under vacuum at ambient temperature for about 18 hours). The resin was removed from the reaction mixture by vacuum filtration through Whatman 54 filter paper. The resin was washed with about one bed volume of dichloromethane and the filtrate was concentrated to approximately 100 ml. The viscous filtrate (now a somewhat cloudy solution) was poured into 1000 ml of hexane to precipitate the polymer. The polymer mass was washed with 2×200 ml of hexane and dried under vacuum. The molecular weight and IV data for the polymers prepared by this process are listed in the table below.

| Sample | Mw (LS), daltons | Mw (CC), daltons | IV, dL/g |
|---|---|---|---|
| 1 | 64,200 | 58,000 | 0.48 |
| 2 | 68,000 | 62,700 | 0.43 |

### EXAMPLE 7

### Synthesis of D,L-PL(PG)EP

All glassware was dried for a minimum of 2 hours at 105° C. and allowed to cool in a desiccator or cooled under a stream of argon gas. A 28.5 g portion of D,L-lactide and 1.5 g of PG (molar ratio, 10: 1) were weighed into a 250 ml 3-neck round-bottom flask. The flask was equipped with a gas joint and a stirrer bearing/shaft/paddle assembly. The mixture was evacuated and filled with argon five times to remove residual air and moisture. The reaction apparatus was immersed in a preheated oil bath at 130° C., connected to an argon source with an oil bubbler, and stirred at a moderate speed until all of the solid monomer had melted.

At this time, a volume of stock stannous octoate solution (about 130 mg/ml in toluene) equivalent to 120 ppm stannous octoate or 35 ppm Sn was added to the melt using a syringe. The reaction mixture was allowed to stir under a slight argon pressure for 4 hours. The oil bath temperature was then reduced to about 110° C. and the residual monomer was removed under vacuum. The upper parts of the reaction assembly were heated gently with a heat gun to aid in the monomer removal. The total time under vacuum was 2 hours.

The molten prepolymer was dissolved in 84 ml of chloroform with stirring and 2.5 equivalents of TEA and 0.5 equivalents of DMAP were added to the stirring reaction mixture using a powder funnel. The reaction mixture was chilled to about −5° C. in a cold bath. A solution of about 1 equivalent of distilled ethyl dichlorophosphonate (EPCl₂) in 9 ml of chloroform was prepared in a dropping funnel. The solution in the funnel was added slowly to the reaction mixture over a period of 0.5 hour. After the addition was complete, the viscosity of the solution had increased significantly and the reaction mixture was allowed to stir at low temperature for 1 hour at −5° C. The reaction was then quenched with 1 ml of anhydrous methanol and stirred for another five minutes.

Next, the reaction mixture was transferred to a 0.5 gallon vessel and mixed with 37 g of Dowex DR-2030 IER and 30 g of Dowex-43, and shaken on a mechanical shaker for 2 hour to remove residual DMAP and TEA free base and salts (the IERs had been washed with several bed volumes of methanol and chloroform and dried under vacuum at ambient temperature for about 18 hours). The resin was removed from the reaction mixture by vacuum filtration through Whatman 54 filter paper. The resin was washed with about one bed volume of dichloromethane and the filtrate was concentrated to approximately 50 ml. The viscous filtrate was poured into 200 ml of petroleum ether to precipitate the polymer. The polymer mass was washed with 100 ml of petroleum ether and dried under vacuum. The molecular weight data for the polymers prepared by this process are listed in the table below.

| Sample | Mw (LS), daltons | Mw (CC), Daltons |
|---|---|---|
| 1 | 339,900 | 327,600 |
| 2 | 369,800 | 360,900 |

### EXAMPLE 8

### Synthesis of P(Cis- and Trans-CHDM/HOP)

All glassware was dried for a minimum of two hours at 105° C. and allowed to cool in a desiccator or cooled under a stream of argon gas. A reaction assembly consisting of a 1 L three-neck round-bottom flask equipped with a gas joint, a stirrer bearing/shaft/paddle and a dropping funnel. A solution of 20.0 g of 1,4-cyclohexane dimethanol (CHDM) was prepared in 75 ml of anhydrous tetrahydrofuran (THF) and transferred to the reaction vessel. The beaker was rinsed with 25 ml of THF and the wash was transferred to the reaction vessel.

Next, 29.0 ml of N-methylmorpholine (NMM) and 1.61 g of DMAP were added to the reaction mixture through a powder funnel. A solution of 28.86 g of hexyl dichlorophosphate (HOPCl₂) in 30 ml of THF was prepared under argon and transferred to the dropping funnel while the reaction mixture was cooled to 4° C. in a cold bath. The solution in the funnel was added to the reaction mixture over a period of one hour. With 5 to 10 minutes after the start of addition, a white precipitate, presumably the hydrochloride salts of NMM and DMAP, began to form. After the addition was complete the funnel was rinsed with 30 ml of THF. The reaction mixture was stirred for 1 hour at 4° C. and then for either 2 or 18 hours at ambient temperature.

At the prescribed time, the precipitate was removed from reaction mixture by vacuum filtration. The filtrate was diluted with 100 ml of dichloromethane, transferred to a half-gallon jar and 86.5 of dried Dowex HCR-S IER and 103.8 g of dried Dowex M-43 IER were added to the filtrate. The jar was sealed with a Teflon lined lid and the mixture was agitated on a mechanical shaker for two hours.

At this time, the IERs were removed by vacuum filtration and the filtrate was concentrated to approximately 100 ml under vacuum. The polymer solution was poured in 2 L of hexane and the resulting fluid material that precipitated was isolated and transferred to a Teflon lined glass dish. The polymer was dried under vacuum to yield a sticky, free flowing viscous liquid. The Mw (LS) data for the polymers prepared by this process are listed in the table below.

| Sample | Mw (LS), daltons | Mw (CC), daltons | IV, dL/g |
|---|---|---|---|
| 1 | 4400 | 5500 | 0.14 |
| 2 | 5000 | 6500 | 0.11 |
| 3 | 4000 | 4600 | 0.10 |

### EXAMPLE 9

### Synthesis of P(BHET/EOP)

All glassware was dried for a minimum of two hours at 105° C. and allowed to cool in a desiccator or cooled under a stream of argon gas. A reaction assembly consisting of a 500 ml three-neck round-bottom flask equipped with a gas joint, a stirrer bearing/shaft/paddle and a dropping funnel. First, 30.0 g of bis(hydroxyethyl) terephthalate (BHET) and 28.83 g of DMAP were added to the reaction vessel using a powder funnel and mixed with 81 ml of THF. The solids were dissolved with stirring and gentle heating using a heat gun.

After all solids had dissolved, the reaction mixture was cooled to 4° C. in a cold bath. A solution of 19.2 g of ethyl dichlorophosphate (EOPCl₂) in 24 ml of THF was prepared in a 125 ml addition funnel. The solution in the funnel was added to the solution in the flask over a period of 1 hour. Shortly after the addition had begun, a white precipitate, presumably DMAP hydrochloride, began to precipitate from the reaction mixture. After all of the solution in the funnel had been added, the stirrer shaft/paddle became entrapped in a thick, stiff precipitate and stirring ceased. It appears the polymer that had formed at this time was insoluble in the reaction mixture.

Next, 125 ml of dichloromethane were added and the reaction mixture was swirled by hand until mechanical stirring could be resumed. The reaction mixture was now a homogenous solution containing a white free flowing powder. The reaction mixture was stirred at 4° C. for one hour. The cold bath was removed and the reaction mixture was allowed to warm to ambient temperature and stirred for 16 hours. At this time, the white precipitate was removed from the reaction mixture by vacuum filtration and the filter cake was washed with 100 ml of dichloromethane.

The resulting filtrate was transferred to a half-gallon jar and treated with 156.92 g of undried Dowex HCR-S IER and 160.92 g of undried Dowex M-43 IER. The resins were washed with 2 bed volumes of methanol and 2 bed volumes of dichloromethane prior to use. The jar was sealed with a Teflon lined lid and shaken on a mechanical shaker for two hours. The resin was removed by vacuum filtration and the filtrate, ˜600 ml, was concentrated to ˜150 ml. The clear solution was poured into 1.2 L of hexane. The thick oil that precipitated was washed with 400 ml of hexane and transferred to a Teflon lined glass dish, dried under vacuum. The molecular weights were determined by GPC were 2200 for Mw (LS) and 2100 for Mw (CC). The value obtained for IV was 0.10 dL/g.

### EXAMPLE 10

### Synthesis of P(BHET-EOP/TC)

All glassware was dried for a minimum of two hours at 105° C. and allowed to cool in a desiccator or cooled under a stream of argon gas. A reaction assembly consisting of a 500 ml three-neck round-bottom flask equipped with a gas joint, a stirrer bearing/shaft/paddle and a dropping funnel. First, 30.0 g of BHET and 28.83 g of DMAP were added to the reaction vessel using a powder funnel and mixed with 81 ml of THF and 125 ml of dichloromethane.

The solids were dissolved with stirring and gentle heating using a heat gun. After all solids had dissolved, the reaction mixture was cooled to 4° C. in a cold bath. A solution of 19.2 g of EOPCl₂ in 24 ml of THF was prepared in a 125 ml addition funnel. The solution in the funnel was added to the solution in the flask over a period of 1 hour. Shortly after the addition had begun, a white precipitate, presumably DMAP hydrochloride, began to precipitate from the reaction mixture. The reaction mixture was stirred at 4° C. for one hour. Next, a solution of 4.79 g of terephthaloyl chloride (TC) in 18 ml of THF was prepared in the addition funnel and added to the solution in the flask over a 30-minute period. The reaction mixture was stirred for one hour at 4 C.

At this time the cold bath was removed and the reaction was allowed to warm to room temperature and stir for another 20 hours. At this time, the white precipitate was removed from the reaction mixture by vacuum filtration. The resulting filtrate was transferred to a half-gallon jar and treated with 88.5 g of dried Dowex HCR-S IER and 73.8 g of dried Dowex M-43 IER. The jar was sealed with a Teflon-lined lid and shaken on a mechanical shaker for two hours. The resin was removed by vacuum filtration and the filtrate was concentrated to ˜100 ml. The clear solution was poured into 2 L of hexane. The thick oil that precipitated was transferred to a Teflon-lined glass dish, dried under vacuum. The molecular weights were determined by GPC were 7200 for Mw (LS) and 4000 for Mw (CC). The value obtained for IV was 0.09 dL/g.

### EXAMPLE 11

### Large-Scale Preparation of D,L-PL(PG)EOP

A 100 g portion of propylene glycol was added to a 3000 ml 3-necked round bottom flask equipped with a gas joint, a stirrer bearing/shaft/paddle assembly, and a Teflon-coated thermocouple. The reaction apparatus was placed in a preheated oil bath at 130° C. and purged with nitrogen for one minute. A 2000 g portion of D,L-lactide was added using a powder addition funnel over a period of 45 minutes. The reaction apparatus was then immersed in the oil so that the oil level was at the bottom of the ground glass joints. The mixture was stirred until all of the solid monomer had melted and the internal temperature had reached approximately 125° C. At this time, a volume of solution of stannous octoate in chloroform equivalent to approximately 400 ppm (117 ppm Sn) was added to the melt using a syringe. The mixture was allowed to stir for approximately 3-16 hours. Then oil bath set point was decreased to approximately 125° C. and any residual unreacted monomer removed using vacuum over approximately 1 hour.

A 2500 ml portion of chloroform was used to dissolve and transfer the prepolymer to a pre-chilled, 20-liter jacketed reactor, which contained 2.5 equivalents (based on propylene glycol) of triethylamine and 0.5 equivalents of DMAP dissolved in 3600 ml of chloroform. The reactor was equipped with a stirrer bearing/shaft/turbine assembly, a gas joint, a tubing adapter, and a Teflon-coated thermocouple. With stirring and chilled recirculation on the jacket, the solution was cooled to below −15° C. A solution of 1 equivalent (based on propylene glycol, approximately 215 g) of distilled ethyl dichlorophosphate (EOPCl₂) in 650 ml chloroform was prepared in a 1000 ml 3-necked round bottom flask equipped with a tubing adapter and a gas joint. The EOPCl₂/chloroform solution was added using a piston pump and Teflon tubing over a period of 50 minutes, maintaining the internal temperature at approximately −10° C. Tubing was connected to the gas joints of the flask and reactor to equalize the pressure during the addition. Following the addition, a 50 ml portion of chloroform was added to rinse the flask, feed lines, and pump. The reaction mixture was stirred for 1 hour at low temperature (−8° C. after 1 hour) before the reaction was quenched with 140 ml of anhydrous methanol.

The reactor was then charged with 3 kg of Dowex DR-2030 IER and 3 kg of Dowex M-43 wetted with approximately 6.5 liters of methylene chloride. The polymer/resin mixture was mixed at low temperature for 3-15 hours, after which it was transferred by vacuum to a stainless steel laboratory Nutsche filter. After filtering off the resin, the polymer solution was pulled through the in-line 8 micron cartridge filter into the concentrator (a similar 10-liter jacketed reactor) where the solution was concentrated with the aid of heated recirculating fluid on the jacket. The 20-liter reactor and the resin in Nutsche were washed with 5 liters of methylene chloride, which were transferred to the concentrator after being stirred for 1 hour. An additional 5 liters of methylene chloride were added to the resin in the Nutsche and added to the concentrator when the solution had been reduced to approximately 6 liters.

Concentration of the polymer solution continued until approximately 4-5 liters of a viscous solution remained. A portion of 1500 ml of ethyl acetate was then added to the polymer solution. The mixture was mixed until homogenous and precipitated in approximately 10 liters of petroleum ether. After the precipitation mixture was stirred for approximately 5 minutes, the supernatant liquid was decanted. The polymer was then washed with 5 liters of petroleum ether. After the mixture was stirred for 5 minutes. The liquid was again decanted. The polymer was poured into a Teflon-coated pan and placed in the vacuum oven at NMT 50° C. After drying for 24 hours, the polymer was ground into smaller pieces and dried for additional time in a vacuum oven at ambient temperature.

### EXAMPLE 12

### Encapsulating Paclitaxel Into the Subject Polymers

The term “PACLIMER” shall refer to a subject polymer in a microsphere form with the D,L-PL(PG)EOP composition containing paclitaxel at certain loading levels. The D,L-PL(PG)EOP polymer in PACLIMER may be prepared using the method described in Example 1, 2 or 11. The loading level of paclitaxel will be expressly stated or alternatively indicated in parentheses as shown for the following examples: for 30% loading level, “PACLIMER (30%)”; for fifty percent loading, “PACLIMER (50%)”; etc. All microspheres of PACLIMER, unless otherwise indicated, were prepared using the solvent dilution method described below.

The four methods listed below may be applied to a variety of drug in polymer loadings:

Method 1—Spray Drying: 10 g of a phosphorous linked polymer, e.g., D,L-PL(PG)EOP, is dissolved in methylene chloride at a concentration of about 10%. After the polymer is completely dissolved, an appropriate amount of paclitaxel powder (e.g., 1.1 g for 10% loading, 4.2 g for 30% loading, 10 g for 50% loading, etc.) is added to the solution and stirred until the powder is completely dissolved. Microspheres are then prepared using a spray-drying technique, e.g., using a Buchi Mini Spray Dryer (Model B-191) at inlet temperature of 35° C., pump rate of 16%(˜10 gm/min) for polymer solution and 800 L/hr for atomizer gas (nitrogen), and aspiration at 50% (˜20 mbar). In most instances, the mean diameter of the resulting microspheres for PACLIMER at various loading levels is less than about 20 microns.

Method II—Solvent Evaporation: Microparticles of the subject compositions will be prepared by solvent evaporation. For example, the subject polymer composition and paclitaxel are dissolved in ethyl acetate, the ethyl acetate solution is then emulsified into a 0.5% polyvinylalcohol (PVA) solution presaturated with ethyl acetate at a stirring rate of 600 rpm, followed by the application of a vacuum (e.g., about 15 inches of Hg) to remove the ethyl acetate. For one exemplary process, the ethyl acetate concentration will be reduced to below 10% within 10 minutes. Microparticles will be washed on an appropriate sieve with deionized water and thereafter lyophilized.

Method III—Solvent Dilution: Microspheres may be prepared by a solvent dilution method using an in-line homogenizer. For example, approximately 50 grams of paclitaxel and 450 grams of subject polymer composition were weighed and dissolved in 1L of ethyl acetate. The non-solvent phase was pre-saturated with ethyl acetate; ethyl acetate (800 ml) was added to 9 liter of 0.5% PVA and homogenized for 1 minute. The paclitaxel-subject polymer composition solution and the PVA -ethyl acetate solution were pumped simultaneously through an in-line homogenizer into a container at rates of 1 and 3 liters/min, respectively. The combined solution was gently stirred with an overhead stirrer. Approximately 90 liters of water was added to the container at a rate of 3 L/min. The solution was then gently stirred for 30 minutes. The microsphere suspension was transferred to a filtering/drying unit containing 150 μm scalping and 25 μm product sieves. The resulting microspheres were rinsed with 5 liters of de-ionized water and dried for 3 days under vibration, vacuum and a nitrogen purge. The dried microspheres on the 25 μm sieve were collected into a container.

Method IV—Freeze/pulverize: Microparticles are prepared by evaporating the drug/polymer in solution at 40° C. under a nitrogen purge to obtain viscous mass which is subsequently cooled to −40° C., lyophilized, e.g., for 48 hours, and pulverized to a desired size for the microparticles.

### EXAMPLE 13

### Animal Efficacy Studies with PACLIMER in Prostate Cancer

To test the efficacy of paclitaxel microsphere formulations in murine and human orthotopic squamous cell prostate cancer model.

The Models:

The LNCaP cell line is the only human prostate cancer cell line established with functional androgen receptor and prostate specific antigen (PSA) expression. Twenty years ago, the LNCaP cell line was derived from a lymph node metastasis from an androgen-responsive prostate tumor from a white male 60 years of age.

The disadvantages of a subcutaneous LNCaP tumor model is that the cells are weakly tumorigenic and thus do not grow well and metastases do not occur. Placing the cells at the appropriate anatomic site provides not only a growth advantage but the tumors are also able to metastasize. Usually, microscopic metastasis can be observed in the retroperitoneal, mediastinal lymph nodes and possibly lung. The control tumors can reach sizes of 1-3 grams but the danger is that the animal dies from renal failure due to blockage of the urethra prior to tumor harvesting. Thus, it is imperative to monitor (visual inspection and PSA serum levels) the mice closely for disease progression and to end the study before premature deaths. Since the model is fairly novel, there is very little data on compounds that are effective in this model. Also, due to technical difficulty, researchers prefer to use alternative easier models.

Protocols:

In vitro:

LNCaP cells are maintained in RPMI 1640, 10% fetal bovine serum, 1% penicillin/streptomycin and 1% L-glutamine. Already published reports state that the IC50 for paclitaxel in the LNCaP cell line is low nM or sub nM.

In vivo:

Male nude (nu/nu) mice were anesthetized and 1×10⁶ LNCaP cells were injected into the ventral prostate. Two weeks following cell injection, PSA serum levels were measured. Depending on the PSA measurements, animals were treated in the following week or two. Mice were randomized into four groups, blank microspheres and PACLIMER (40%) injected subcutaneous or intratumorally (20 gauge needle). Tumors were approximately 200 mm³ on day of injection.

Dose Groups: (n=8/9 per group)

| | |
|---|---|
| Placebo i.t. | 240 mg/kg blank microspheres of D,L-PL(PG)EOP |
| | used to make PACLIMER (60 mg/ml injected 100 μl) |
| PACLIMER i.t. | 240 mg/kg high dose PACLIMER (40%) (60 mg/ml |
| | injected 100 μl - 24 mg paclitaxel delivered) |
| Placebo s.c. | 50 mg/ml delivered 0.5 ml = 25 mg blank |
| | microspheres of D,L-PL(PG)EOP used |
| | to make PACLIMER |
| PACLIMER s.c. | 50 mg/ml delivered 0.5 ml = 25 mg PACLIMER |
| | (40%) (10 mg paclitaxel delivered) |

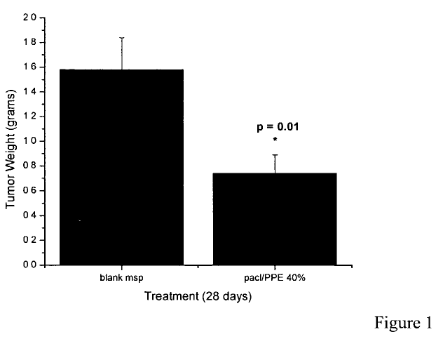

A significant anti-tumor effect was observed when PACLIMER (40%) was delivered intratumorally (Table 1; FIG. 1). Tumor size measured by weight decreased from 1.59±0.26 grams in the placebo group to 0.74±0.16 grams in the treated group (p=0.01). Treated/Control ratio (T/C) is 0.47.

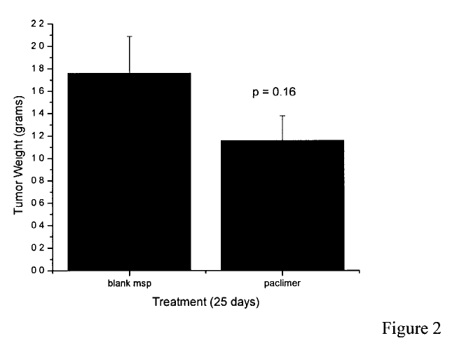

In the s.c. treated animals, a trend to tumor reductions was observed with PACLIMER (40%) (Table 1; FIG. 2). Tumor size measured by weight decreased from 1.75±0.33 g in the placebo group to 1.16±0.22 g in the treated group (p=0.16).

TABLE 1
Effect of PACLIMER (40%) on orthotopic LNCaP prostate cancer

| Treatment | Tumor Size (grams ± se) | P value |
|---|---|---|
| Intratumoral | | |
| Placebo msp | 1.59 ± 0.26 | P = 0.01 |
| PACLIMER (40%) | 0.74 ± 0.16 | |
| Subcutaneous | | |
| Placebo msp | 1.75 ± 0.33 | P = 0.16 |
| PACLIMER (40%) | 1.16 ± 0.22 | |

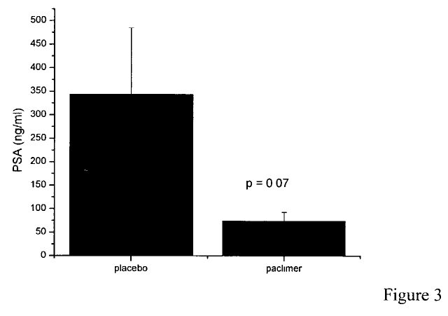

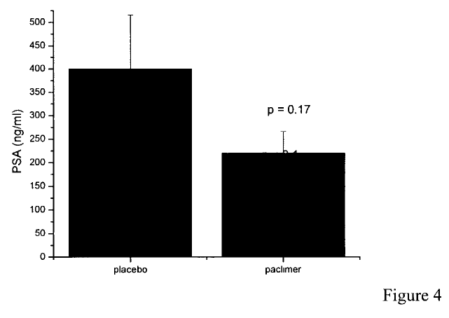

Serum PSA levels were measured prior to treatment and at the end of the study. The purpose of measuring PSA is to determine if PSA can be used as a surrogate marker for effectiveness of treatment. There was a clear difference in PSA levels between treated and placebo but due to high variance in raw data, no significant difference was observed (FIGS. 3 and 4).

In Combination with Electromagnetic Radiation

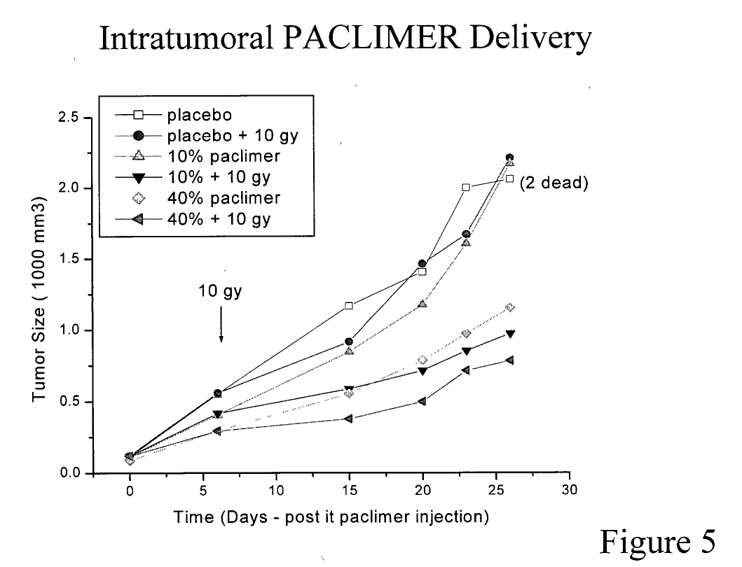

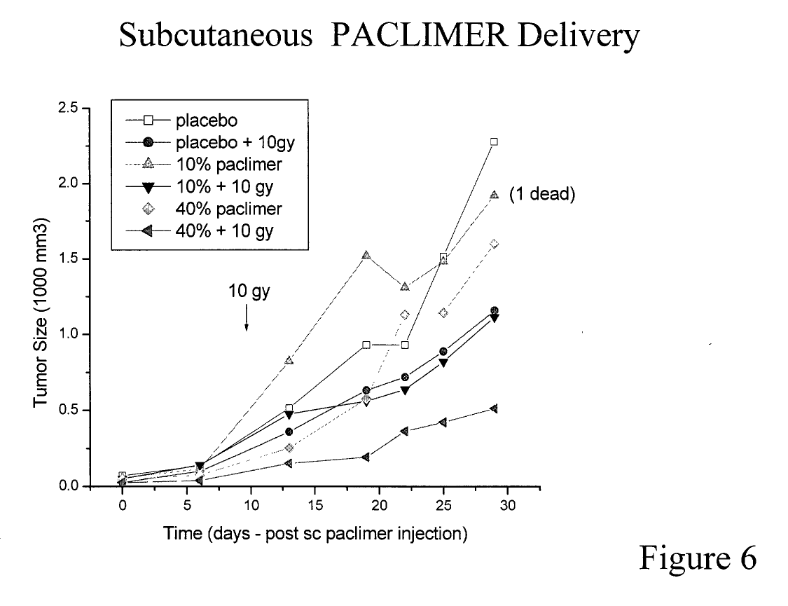

In these experiments, PACLIMER treatment, both 10% and 40% loading with paclitaxel, was combined with radiation therapy. The model was a xenograft or flank tumor grown from a human prostate cancer cell line called TsuPr1. PACLIMER (either 10% or 40%) was injected either intratumorally (FIG. 5) or subcutaneously (FIG. 6) on the contralateral flank as the tumor. Radiation was given as 10 gy as one dose one week post-PACLIMER injection. Three week post cell injection the tumors were injected intratumorally with PACLIMER and then followed by fractionated radiation, 2 gy/day for 4 days. Results are shown in FIGS. 5 and 6.

### EXAMPLE 14

### Clinical Treatments for Prostate Cancers

For prostate cancer clinically confined to the prostate itself, surgery or radiation are available to extirpate the disease. In early stage disease, these methods of local treatment are generally effective, so that the patient may be monitored after the initial intervention for evidence of distant spread or recurrent disease and treated appropriately when such evidence presents itself. Surgical management of early stage disease typically involves a radical prostatectomy, performed via a retropubic or a perineal approach. A modification of the standard technique may permit sparing of the cavernous nerves located posterolateral to the prostate in close association with the lateral prostatic fascia and rectum, these nerves being understood to carry autonomic innervation to the penis and particularly to the corpora cavernosa. Preservation of these nerves increases the likelihood that erectile capacity will be retained after surgery. More extensive disease may still require radical prostatectomy for local control, with the possible addition of neoadjuvant or adjuvant systemic therapy. Prostate cancer that has extended beyond the prostatic capsule poses a greater risk for local recurrence or systemic spread, both locoregionally and metastatically. In certain instances, sampling or removal of the regional lymph nodes may be recommended in combination with radical prostatectomy to reduce the burden of locoregional disease and to inhibit its potential for further dissemination. More extensive surgical resections increase the risk of perioperative complications, including bleeding, infection, impotence, incontinence and other local or systemic complications. Radiation may supplant surgery in the treatment of early tumors, or may offer local control for more advanced tumors inappropriate for surgical resection. Additionally, radiation may be combined with surgery in the treatment of more advanced disease and radiation can be used for salvage of local recurrence in a patient previously treated with radical prostatectomy. When the patient recurs locally or locoregionally, the goals of treatment shift to palliation and salvage. It is understood that surgery in the salvage context is associated with increased morbidity.

In one embodiment of the present invention, a subject composition may be positioned locally in an anatomic area following a surgical resection. The composition may be shaped as a film, a mesh, a solid article, a spray, or any other form that is adaptable to the location and dimensions of the extirpation site. Alternatively, the composition may be injected into the margins of the resection bed. As another alternative, the composition may be applied to the surfaces or the substance of organs or structures being preserved in a surgical site. For example, a film comprising a subject composition may be used to wrap the nerves or vessels exposed during a radical prostatectomy dissection to provide a barrier for tumorous ingrowth, thus potentially decreasing the likelihood of subsequent malignant involvement of these structures. Since the presence of positive surgical margins has been correlated with increased disease relapse, control of the surgical margin is desirable (Ohori M, Wheeler T M et al, “Prognostic significance of positive surgical margins in radical prostatectomy specimens,” J. Urol. 154:1818, 1995) Although modifications of surgical techniques may reduce the incidence of positive surgical margins, control of the surgical margin remains a concern in the art (Klein E A, Capelin P A et al, “Initial dissection of the lateral fascia reduces the positive margin rate in radical prostatectomy,” Urol. 51:766, 1998). A composition according to the present invention may be placed in the anatomic region of the resected prostate to counteract certain adverse effects of a positive surgical margin, or to reduce the probability of leaving viable cancer cells in place at a positive surgical margin. In certain cases, there is demonstrated or suspected perineural invasion of the cavernous nerves, generally treated with excision of the neurovascular bundles and subsequent compromise of erectile capacity (Holmes G F, Walsh P C et al, “Excision of the neurovascular bundle at radical prostatectomy in cases with perineural invasion on needle biopsy,” Urology 53:752, 1999) When used in the context of nerve-sparing surgery, protecting the exposed nerves with devices utilizing the present invention may enhance likelihood of preserved erectile function. The availability of the present invention as an adjunct to surgical resection may permit nerve sparing surgery to be performed in situations where more extensive disease previously would have foreclosed this option. Recognizing that susceptibility of tumor cells to antineoplastic substances may be diminished in the immediate postoperative phase, a composition according to the present invention may be constructed so that the antineoplastic substance is released on a time-release basis according to a preselected timetable so that its efficacy will correspond to the biology of maximum tumor susceptibility. In certain embodiments, compositions according to the present invention may be delivered in a form whereby they are retained locally despite the need for closed suction drainage of the surgical wound. In other embodiments, compositions according to the present invention may be combined with other therapeutic agents to address local wound conditions such as bacterial contamination, tendency for tissue fluid collection, or decreased likelihood of wound healing. Other combinations and embodiments will be appreciated by those of ordinary skill in the art.

Certain embodiments of the present invention will be suitable for injection directly into a tumor, into a tumor bed, or into the periphery surrounding a resected, partially resected or unresected tumor. In other embodiments, subject compositions according to the present invention may be infused into blood vessels supplying a tumor using techniques of local infusion, superselective arteriography, or other techniques familiar to practitioners in the relevant arts. In yet another embodiment, subject compositions may be formulated as implants to be inserted into the prostate gland in preselected locations. The compositions may be formulated as needle-implantable “seeds” or other implantable devices that can be localized locally or diffusely within the prostate gland using positioning techniques analogous to those employed with brachytherapy. In certain cases, this type of implantation technique may be combined with brachytherapy or external beam radiation or other forms of radiation therapy, as will be understood by those of ordinary skill in the art. The systems and methods of the present invention may be used to treat large primary tumors, or may be used to treat residual tumors following radiation or chemotherapy, or may be used to treat recurrent disease. It is understood that under certain circumstances encompassed by the scope of the present invention, these agents may be used in combination with other treatment modalities. For example, a recurrence identified by MRI may be treated by intra-tumoral injection alone or in combination with radiotherapy if tissues can tolerate it; thereafter, surgical means may be employed to resect the gross disease, such disease having been diminished in volume by the delivered treatment modalities. As another example, the compositions of the present invention may be used in combination with other agents, such as radiosensitizers, that are adjunctive to radiation therapy treatments, whether primary radiation therapy or radiation therapy for recurrent disease. In one embodiment, the compositions of the present invention may be implanted into the prostate tumor or prostate gland prior to administration of radiation, either primarily or for recurrence; in this case, radiosensitizing agents may optionally be used as well. The use of radiosensitizing agents has been disclosed in U.S. patent application Ser. No. 09/976,283, the contents of which is herein incorporated by reference. In another embodiment, the compositions of the present invention may be implanted into the prostate tumor or prostate gland following the administration of radiation, either primarily or for recurrence.

The systems and methods of the present invention are suitable for utilization in conjunction with primary radiation of the prostate for cancer control, where either brachytherapy or external beam irradiation is used. A polymer bearing antineoplastic substances according to the present invention may injected into the glandular tissue using techniques familiar to practitioners of ordinary skill in the art. Variations of standard practices and delivery systems may, with no more than routine experimentation, be envisioned by skilled artisans to enable instillation of the composition of the present invention into affected tissues. For example, an intraurethral delivery system may be contemplated to permit the transurethral injection, instillation or other delivery of a polymer bearing antineoplastic agents into the prostate gland. After the composition of the present invention are delivered into the prostate, the organ may be treated with a therapeutic dose of radiation. Alternatively, if radiation-containing implants are inserted into the gland, using for example the techniques of brachytherapy, compositions according to the present invention may be delivered to the prostate preceding the brachytherapy, concurrent with the brachytherapy or following such treatment. In certain embodiments, subject compositions may be fabricated so as to be combined with a deliverable radiation source, for example a radiation seed as would be used in brachytherapy. In these embodiments, the positioning step of brachytherapy would insert seeds in preselected areas of the prostate gland that would deliver both the desired therapeutic radiation and an antineoplastic agent. In certain embodiments, the compositions of the present invention may act as radiation sensitizers, so that a lower dose of radiation may be used for primary or recurrent disease, or so that more extensive tumor extirpation may be accomplished with standard radiation doses.

The systems and methods of the present invention may be used to supplement surgical dissections of the regional lymph node structures, whether performed in conjunction with the radical prostatectomy or undertaken as a separate procedure, for example laparoscopically. For example, a composition according to the present invention may be placed in the surgical bed following prostatectomy and lymph node dissection, or may be placed in a dissection bed following laparoscopic lymphadenectomy. Such a composition may take a form of a fluid, a flowable substance, a flexible article such as a sheet or a mesh, or any other conformable device suitable for placement in a surgical bed and adapted for remaining in a preselected position therein. Substances according to the present invention may also be instilled under the wound closure flap using the drainage catheters placed there for routine surgical drainage. Use of systems and methods according to the present invitation may further be used to treat regional lymph nodes prophylactically without needing to resect them in cases where a risk of regional lymph node involvement exists without this involvement having been anatomically determined. Such use of the systems and methods of the present invention may be appropriate, for example, for a tumor clinically confined to the prostate gland but having a high pathological grade or other risk factor associated with it.

Formulations of the present invention may be adapted for use in as part of a strategy for salvage after local recurrence. For example, a composition according to the present invention may be injected into or otherwise delivered to an area of recurrent disease that has been identified clinically or radiologically. Precise positioning of the polymeric substance may be accomplished using minimally invasive techniques directed by ultrasound, MRI or other radiologic modalities. Recognizing the palliative purpose of salvage procedures, a practitioner may elect to treat a local recurrence solely using such local administration of a device according to the present invention, or may use the present invention in conjunction with other salvage procedures such as surgery or radiation.

Local delivery of antineoplastic agents using the systems and methods of the present invention may under certain circumstances be useful in conjunction with systemic antineoplastic treatment. Although systemic antineoplastic agents have a low rate of efficacy for prostate adenocarcinoma, use of the present invention to deliver locally directed doses of a therapeutic agent may act synergistically with systemic chemotherapy in the treatment of extensive local or locoregional disease. In other embodiments, the systems and methods of the present invention may be adapted for treatment of distant metastases that can be isolatable. For example, application of a subject composition to a discrete area of bony involvement with metastatic disease may permit sustained release of high dose antineoplastic treatment to the metastasis with clinical benefit, including symptomatic palliation. Furthermore, compositions according to the present invention may be formulated to provide structural support in addition to delivering antineoplastic treatment, a feature that may be useful in cases of bony involvement where pathological fracture is a risk.

These examples of the clinical utility of the present invention have been provided for illustrative purposes only. Other exemplary utilizations will be apparent to practitioners of ordinary skill in the art using no more than routine experimentation. For example, the systems and methods of the present invention, while being illustrated with reference to the prostate gland, may be suitable for use with other solid or hollow organs. Other organs, such as the thyroid, the parathyroid, the salivary gland, the pancreas, the kidney or the adrenal gland may, when afflicted with a neoplasm, be treated with the compositions of the present invention before definitive extirpative surgery or radiation treatment is undertaken. Following the examples provided herein, the compositions of the present invention may be placed within a solid organ prior to definitive treatment in those cases where the tumor is too large or precariously placed to be readily treated with surgery. The compositions of the present invention may also provide palliation in those cases where definitive treatment is not possible due to the extent of disease in the solid organ. Compositions according to the present invention may also be used in the walls of hollow viscus organs in circumstances where pre-treatment before definitive surgery might be desirable, or in circumstances where extensive disease precludes definitive curative therapy. For example, in cancers of the bladder, there may be extensive disease affecting the wall of the organ that may be effectively treated by implanting devices bearing the compositions of the present invention within the bladder wall. Implantation of devices bearing compositions of the present invention may be also carried out to palliate an advanced malignancy in an organ difficult to treat with conventional surgery, for example the pancreas, with the intention of shrinking the tumor size and slowing its spread into adjacent organs. In certain embodiments, compositions formulated according to the present invention may be suitably instilled in advanced disease using minimally invasive means such as endoscopy, thus sparing the patient more extensive surgery that lacks curative potential. While these examples have been provided to illustrate more clearly the features and principles of the present invention, it is understood that other application of these features and principles will be apparent to those of ordinary skill in the relevant arts with the use of no more than routine experimentation.

9. Equivalents

Those skilled in the art will recognize, or will be able to ascertain using no more than routine experimentation, many equivalents to the specific embodiments and practices of the invention described herein. Such equivalents are intended to be encompassed by the following claims.

We claim:

1. A method for treating prostrate cancer of a patient, comprising: instilling into an anatomic area of a patient affected by said prostrate cancer a therapeutically effective amount of a composition comprising a biocompatible polymer and an antineoplastic agent, wherein said polymer comprises phosphorous-based linkages.

2. The method of claim 1, wherein said polymer is biodegradable.

3. The method of claim 1, wherein said antineoplastic agent is an antineoplastic taxane.

4. The method of claim 3, wherein said antineoplastic taxane is paclitaxel.

5. The method of claim 1, wherein said polymer comprises one or more monomeric units represented by the following formula V: 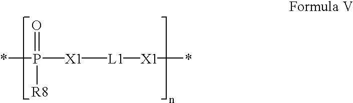
wherein, independently for each occurrence of said monomeric unit:
X1, each independently, represents —O— or —N(R7)-;
R7 represents —H, aryl, alkenyl or alkyl;
L1 represents any chemical moiety that does not materially interfere with the biocompatibility of said polymer;
R8 represents —H, alkyl, —O-alkyl, —O-cycloalkyl, aryl, —O-aryl, heterocycle, —O-heterocycle, or —N(R9)R10;
R9 and R10, each independently, represent a hydrogen, an alkyl, an alkenyl, —(CH2)m-R11, or R9 and R10, taken together with the N atom to which they are attached complete a heterocycle having from 4 to about 8 atoms in the ring structure;
m represents an integer in the range of 0-10; and
R11 represents —H, alkyl, aryl, cycloalkyl, cycloalkenyl, heterocycle or polycycle.

6. The method of claim 1, wherein said prostrate cancer is an adenocarcinoma.

7. The method of claim 1, wherein an access device is used for said instillation.

8. The method of claim 1, further comprising treating said patient with radiation.

9. The method of claim 8, wherein said radiation comprises external beam radiation.

10. The method of claim 8, wherein said radiation comprises brachytherapy.

11. The method of claim 8, wherein at least a plurality of said radiation treatment occurs after instillation of said composition.

12. The method of claim 8, wherein at least a plurality of said radiation treatment occurs before instillation of said composition.

13. The method of claim 8, wherein said radiation treatement occurs before and after said instillation of said composition.

14. The method of claim 1, wherein said polymer comprises one or more monomeric units represented by the following formula VI: 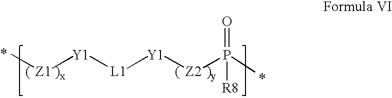
wherein Z1 and Z2, respectively, for each independent occurrence is: 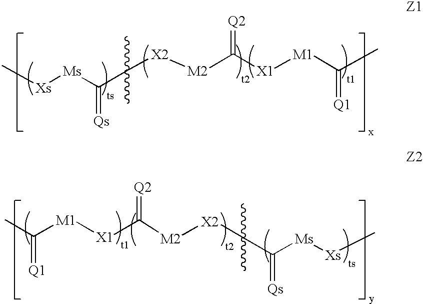
wherein, independently for each occurrence of said monomeric unit:
Q1, Q2 . . . Qs, each independently, represent —O—O or —N(R7);
X1, X2 . . . Xs, each independently, represent —O— or —N(R7);
R7 represents —H, aryl, alkenyl or alkyl;
the sum of t1, t2 . . . ts is an integer and equal to at least one or more;
Y1 represents —O—, —S— or —N(R7)-;
x and y are each independently integers from 1 to about 1000 or more;
L1 represents any chemical moiety that does not materially interfere with the biocompatibility of said polymer;
M1, M2 .. Ms each independently, represents any chemical moiety that does not materially interfere with the biocompatibility of said polymer;
R8 represents —H, alkyl, —O-alkyl, —O-cycloalkyl, aryl, —O-aryl, heterocycle, —O-heterocycle, or —N(R9)R10;
R9 and R10, each independently, represent a hydrogen, an alkyl, an alkenyl, —(CH2)m-R11, or R9 and R10, taken together with the N atom to which they are attached complete a heterocycle having from 4 to about 8 atoms in the ring structure;
m represents an integer in the range of 0-10; and
R11 represents —H, alkyl, aryl, cycloalkyl, cycloalkenyl, heterocycle or polycycle.

15. The method of claim 1, wherein said composition is formulated as microspheres.

16. The method of claim 1, wherein said composition is in the form of microparticles.

17. The method of claim 1, wherein said polymer comprises one or more monomeric units represented by the following formula VII: 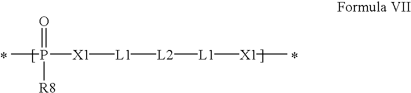
wherein, independently for each occurrence of said monomeric unit:
X1, each independently, represents —O— or —N(R7)-;
R7 represents —H, aryl, alkenyl or alkyl;
L1 represents any chemical moiety that does not materially interfere with the biocompatibility of said polymer;
R8 represents —H, alkyl, —O-alkyl, —O-cycloalkyl, aryl, —O-aryl, heterocycle, —O-heterocycle, or —N(R9)R10;
R9 and R10, each independently, represent a hydrogen, an alkyl, an alkenyl, —(CH2)m-R11, or R9 and R10, taken together with the N atom to which they are attached complete a heterocycle having from 4 to about 8 atoms in the ring structure;
m represents an integer in the range of 0-10, preferably 0-6; and
R11 represents —H, alkyl, aryl, cycloalkyl, cycloalkenyl, heterocycle or polycycle; and
L2 represents a divalent, branched or straight chain aliphatic group, a divalent cycloaliphatic group, a phenylene group, or a group of the formula:

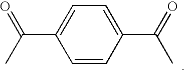

18. The method of claim 1, wherein said method provides extended release of said antineoplastic agent into said anatomic area.

19. The method of claim 1, wherein a portion of said composition is injected intratumorally into tumors of said prostrate cancer.

20. The method of claim 1, wherein said method increases the median survival rate from said prostrate cancer by at least about 10 percent as compared with the median survival rate obtained by administration of substantially the same effective dosage of said antineoplastic agent not incorporated in said composition.

21. The method of claim 1, wherein said method increases the median survival rate for a five year period from said prostrate cancer by at least about 25 percent as compared with the median survival rate obtained by administration of substantially the same effective dosage of said antineoplastic agent without said polymer.

22. The method of claim 21, wherein said antineoplastic agent is paclitaxel and said antineoplastic agent without said polymer is formulated in 50 percent CREMOPHOR EL and 50 percent dehydrated alcohol.

23. The method of claim 1, wherein said method increases the median survival rate for a three year period from said prostrate cancer by at least about 50 percent as compared with the median survival rate obtained by administration of substantially the same effective dosage of said antineoplastic agent formulated in a pharmaceutically acceptable carrier.

24. The method of claim 1, wherein said method is at least about 75 percent more effective in treating said prostrate cancer than administration of substantially the same effective dosage of said antineoplastic agent formulated in a pharmaceutically acceptable carrier without said polymer.

25. The method of claim 1, wherein said method reduces the number of hypersensitivity reactions obtained upon administration of said composition by at least about 10 percent as compared with the number of hypersensitivity reactions obtained by administration of substantially the same effective dosage of said antineoplastic agent formulated in a pharmaceutically acceptable carrier and without premedication.

26. The method of claim 1, wherein said method reduces the number of significant hypersensitivity reactions obtained by administration of said composition by at least about 25 percent as compared with the number of hypersensitivity reactions obtained by administration of substantially the same effective dosage of said antineoplastic agent not incorporated in said polymer.

27. The method of claim 1, wherein said antineoplastic agent is an antineoplastic taxane, and wherein said method reduces the number of hypersensitivity reactions obtained by administration of said composition by at least about 50 percent as compared with the number of hypersensitivity reactions obtained by administration of substantially the same effective dosage of said antineoplastic taxane formulated in a pharmaceutically acceptable carrier.

28. The method of claim 1, wherein said polymer comprises one or more monomeric units represented by the following formula VIII: 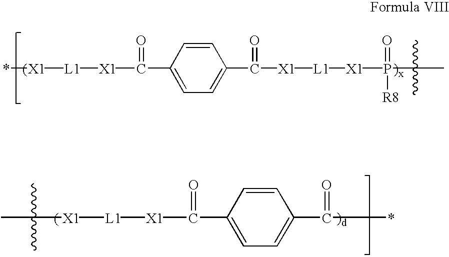
wherein, independently for each occurrence of said monomeric unit:
X1, each independently, represents —O— or —N(R7)-;
R7 represents —H, aryl, alkenyl or alkyl;
L1 represents any chemical moiety that does not materially interfere with the biocompatibility of said polymer;
R8 represents —H, alkyl, —O-alkyl, —O-cycloalkyl, aryl, —O-aryl, heterocycle, —O-heterocycle, or —N(R9)R10;
R9 and R10, each independently, represent a hydrogen, an alkyl, an alkenyl, —(CH2)m-R11, or R9 and R10, taken together with the N atom to which they are attached complete a heterocycle having from 4 to about 8 atoms in the ring structure;
m represents an integer in the range of 0-10, preferably 0-6;
R11 represents —H, alkyl, aryl, cycloalkyl, cycloalkenyl, heterocycle or polycycle; and
d is equal to one or more and x is equal to or greater than one.

29. The method of claim 1, wherein said method releases a therapeutically effective amount of said antineoplastic agent over about at least seven days after said instillation.

30. The method of claim 1, wherein said method releases a therapeutically effective amount of said antineoplastic agent over at least about thirty days after said instillation.

31. The method of claim 1, wherein said method releases a therapeutically effective amount of said antineoplastic agent over about at least sixty days after said instillation.

32. The method of claim 1, wherein said method releases a therapeutically effective amount of said antineoplastic agent over about at least ninety days after said instillation.

33. A composition, comprising: a biocompatible polymer and a therapeutically effective amount of an antineoplastic agent, wherein said composition is suitable for administration to a patient, said composition is in at least partial contact with an anatomic area affected with prostrate cancer, and wherein said biocompatible polymer comprises phosphorous-based linkages.

34. The composition of claim 33 wherein said antineoplastic agent is an antineoplastic taxane.

35. The composition of claim 34, wherein said antineoplastic taxane is paclitaxel.

36. A method for treating a neoplasm located in or around an organ of a patient, comprising: instilling into said organ of said patient affected by said neoplasm a therapeutically effective amount of a composition comprising a biocompatible polymer and an antineoplastic agent, wherein said polymer comprises phosphorous-based linkages.

37. The method of claim 36, wherein said organ is one of the following: thyroid, parathyroid, salivary gland, pancreas, kidney or adrenal gland.

38. The method of claim 36, wherein said organ is hollow.

39. The method of claim 36, wherein said organ is solid.

40. A kit containing a drug delivery system, comprising the composition of claim 33 and instructions for use.
